(19) United States
(12) Patent Application Publication
(10) Pub. No.: US 2026/0199522 A1
(43) Pub. Date: Jul. 16, 2026

(54) AAV VARIANTS FOR THE TREATMENT OF DRY AMD

(71) Applicant: Adverum Biotechinologies, Inc., Redwood City, CA (US)

(72) Inventors: Ruslan Grishanin, Redwood City, CA (US); Gustavo De Alencastro, Redwood City, CA (US); Brigit Riley, Redwood City, CA (US)

(21) Appl. No.: 19/133,100

(22) PCT Filed: Nov. 28, 2023
(86) PCT No.: PCT/US2023/081299

§ 371 Date: May 27, 2025

Related U.S. Application Data
(60) Provisional application No. 63/428,291, filed on Nov. 28, 2022.
(60) Provisional application No. 63/431,537, filed on Dec. 9, 2022.
(60) Provisional application No. 63/465,110, filed on May 9, 2023.
(60) Provisional application No. 63/545,247, filed on Oct. 23, 2023.

(51) Int. Cl. A61K 48/00; A61K 9/00; A61K 38/17; A61P 27/02; C07K 14/005; C12N 15/86

(52) U.S. Cl. CPC A61K 48/005; A61K 9/0048; A61K 38/1725; A61K 48/0075; A61K 48/0083; A61P 27/02; C07K 14/005; C12N 15/86; C12N 2750/14122; C12N 2750/14143; C12N 2750/14145; C12N 2800/22; C12N 2830/15; C12N 2830/42; C12N 2830/50

(57) ABSTRACT

Provided is the intravitreal administration of recombinant adeno-associated virus (rAAV)-based gene therapies for the amelioration of dry-AMD related symptoms, prevention of dry-AMD and treatment of dry-AMD.

### CROSS REFERENCE TO RELATED APPLICATIONS

This application claims priority under 35 U.S.C. § 119(e) to U.S. Provisional Application No. 63/428,291,723 filed Nov. 28, 2022, U.S. Provisional Application No. 63/431,537 filed Dec. 9, 2022, U.S. Provisional Application No. 63/465,110 filed May 9, 2023 and U.S. Provisional Application No. 63/545,247 filed Oct. 23, 2023; each of which is incorporated by reference in its entirety.

### STATEMENT REGARDING THE SEQUENCE LISTING

The official copy of the Sequence Listing is submitted concurrently with the specification as an WIPO Standard ST.26 formatted XML file with file name “17234-041WO1_SequenceListing.xml”, a creation date of Nov. 27, 2023, and a size of 28 kilobytes. This Sequence Listing filed via USPTO Patent Center is part of the specification and is incorporated in its entirety by reference herein.

### FIELD OF INVENTION

The present invention relates to methods of treating dry age-related macular degeneration (Dry AMD) that comprises administering intravitreally a dose of recombinant adeno-associated virus (rAAV) particles encoding a human Complement Factor I (hCFI) gene product to an eye of an individual.

### BACKGROUND

Complement Factor I (also known as Factor I, CFI, and C3b/C4b inactivator) is a protein that, in humans, is encoded by the CFI gene. CFI is post-translationally processed by N-linked glycosylation and furin cleavage prior to translocation facilitated by an 18-residue signal peptide that is cleaved upon secretion. Furin is required to cleave at Arg339 of the RRKR linker of the mature human protein. Incomplete cleavage of the RRKR linker by furin can result in secretion of pro-I (pro-CFI) in addition to mature CFI. CFI is a serine protease that circulates in a zymogen-like state typically at a concentration of about 35 μg/mL (Roversi et al (2011) PNAS 108:12839-12844, Nilsson et al (2011) Mol Immunol 48:1611-1620). CFI inactivates C3b by cleaving it into iC3b, C3d and C3d,g and in an analogous way, C4b into C4c and C4d. Thus, CFI activity downregulates complement cascade in all complement pathways (alternative, classical and lectin). CFI requires the presence of one or more cofactor proteins to perform its functions; cofactor proteins include, but are not limited to, C4BP, CFH, CR1 (also known as CR1/CD35) and MCP (CD46); see Degn et al (2011) Am J Hum Genet 88:689-705. Once C3b has been cleaved into iC3b; iC3b does not perpetuate amplification of the complement cascade or activation through the alternative pathway. iC3b promotes a proinflammatory action by activating complement receptor 3 (CR3) on certain cell types. CFI is capable of processing iC3b into C3dg in the presence of the cofactor CR1. C3dg is unable to bind CR3. C3b binding to CR3 is involved with complement activation leading to inflammation; the breakdown of iC3b to C3dg reduces complement-induced inflammation (Lachmann (2009) Adv. Immunol. 104:115-149). CFI is capable of processing iC3b into an inactive degradation product.

Age-related macular degeneration (AMD) is a degenerative ocular disease affecting the macula, a light sensitive, small area in the center of the retina that is responsible for reading and high acuity. Conditions affecting the macula reduce central vision while leaving peripheral vision intact. In severe cases, the disease can lead to central blindness. AMD is a notable cause of vision loss in the US population among persons 65 years and older, and the estimated prevalence of any AMD among persons over 40 years of age is approximately 6.5% (Klein et al., (2011) Arch Ophthalmol. 129(1):75-80).

The clinical progression of AMD is characterized by stage according to changes in the macula. The hallmark of early AMD is the appearance of drusen, which are accumulations of extracellular debris underneath the retina and appear as yellow spots in the retina during clinical examination and on fundus photographs. Drusen are categorized by size as small (<63 μm), medium (63-124 μm) and large (>124 μm). Drusen are also considered as hard or soft depending on the appearance of their margins on ophthalmological examination. Hard drusen have clearly defined margins; soft drusen have less defined, fluid margins. The Age-related Eye Disease Study (AREDS) fundus photographic severity scale is one of the main classification systems used for this condition.

There are two forms of age-related macular degeneration, dry (atrophic) and wet macular degeneration. Dry-AMD is more common than wet-AMD, but the dry can progress to wet-AMD. Dry-AMD is characterized by thinning of the tissues of the macula as cells disappear; dry-AMD may affect both eyes. Dry AMD is typically characterized by progressive apoptosis of the cells in the retinal pigment epithelium (RPE) layer, overlying photoreceptor cells, and frequently also the underlying cells in the choroidal capillary layer. Confluent areas of RPE cell death accompanied by overlying photoreceptor atrophy are referred to a geographic atrophy (GA). As dry-AMD progresses and GA increases, central vision slowly worsens and the ability to see fine detail is gradually lost. Dry AMD tends to progress more slowly than wet AMD.

Previous efforts to address Dry AMD have involved ocular injections of different compounds to inactivate the complement pathway, such as inhibitors of C3, C5, HtrA1, C1qm, and also natural inhibitors of the complement pathway, such as CFI, CFH and CD59. There is no currently approved medical treatment for dry-AMD.

### SUMMARY

The application provides polynucleotide cassettes for increasing the concentration of human CFI in the vitreous humor and/or retinal extracellular space of a mammalian eye. Also provided are methods of increasing the concentration of human CFI in the vitreous humor and/or retinal extracellular space of a mammalian eye and methods of treating ocular disorders such as dry-AMD and geographic atrophy by administering a vector comprising a polynucleotide cassette for increasing the concentration of human CFI in the vitreous humor and/or retinal extracellular space.

In an embodiment, the application provides a polynucleotide cassette for increasing the concentration of human CFI in the vitreous humor and/or retinal extracellular space of a mammalian eye. Polynucleotide cassettes of the application comprise an enhancer and promoter region, a chimeric intron, a Kozak sequence, a unique coding sequence optimized for increased expression and low CpG, operatively linked to the promoter region wherein the coding sequence is a human CFI gene, a human scaffold attachment region and a polyadenylation site. In an aspect, a polynucleotide cassette of the application further comprises an enhancer element. In various aspects, the chimeric intron comprises at least one element selected from the group comprising (a) an adenovirus tripartite leader sequence (TP), (b) an enhancer element and (c) an intron from mouse IgH. The polynucleotide cassette may further comprise one or more AAV2 inverted terminal repeats (ITRs). In an aspect, the polyadenylation site of the cassette is the human growth hormone polyadenylation site. In various aspects, a polynucleotide cassette of the application has a nucleotide sequence having at least 90% identity to the nucleotide sequence set forth in SEQ ID NO:1. In certain aspects of a polynucleotide cassette of the application, the coding sequence of the human CFI gene is selected from the group consisting of the nucleotide sequences set forth in SEQ ID NO:2, SEQ ID NO:3, SEQ ID NO:4, SEQ ID NO:5 and SEQ ID NO:6.

In an embodiment, the application provides a method of increasing the concentration of human CFI in the vitreous humor and/or retinal extracellular space of a mammalian eye comprising administering to a subject a recombinant adeno-associated virus (rAAV) vector at a dosage ranging from about 1E9 to about 4E12 vector genomes (vg)/eye, wherein the rAAV vector comprises an AAV2 capsid variant and wherein the rAAV vector comprises a polynucleotide cassette. The polynucleotide cassette comprises an enhancer and promoter region, a chimeric intron, a Kozak sequence, a unique coding sequence optimized for increased expression and low CpG, operatively linked to the promoter region wherein the coding sequence is a human CFI gene, a human scaffold attachment region and a polyadenylation site. In aspects of the embodiment, the polynucleotide cassette further comprises an enhancer element. In various aspects of the embodiment, the chimeric intron of the polynucleotide cassette comprises at least one element selected from the group comprising (a) an adenovirus tripartite leader sequence (TPL), (b) an enhancer element and (c) an intron from mouse IgH. In aspects of the method, the polynucleotide cassette further comprises AAV2 inverted terminal repeats. In some aspects, the polyadenylation site of the polynucleotide cassette is the human growth hormone polyadenylation site. In certain aspects, the polynucleotide cassette has a nucleotide sequence having at least 90% identity to the nucleotide sequence set forth in SEQ ID NO:1. In some aspects, the coding sequence of a human CFI gene is selected from the group consisting of the SEQ ID NO:2, SEQ ID NO:3, SEQ ID NO:4, SEQ ID NO:5 and SEQ ID NO:6.

In various aspects of the method, the rAAV vector is administered intravitreally. In some aspects, the AAV2 capsid variant is selected from the group comprising AAV2.7m8 and AAV2.5T-LSV1. In some aspects of the method, the rAAV vector is administered at a dosing range selected from the group of ranges comprising from about 1E9 to about 4E12 vg/eye, from about 10E10 to about 4E11 vg/eye, from about 2E10 to about 2E11 vg/eye and from about 2E10 to about 1E11 vg/eye. In certain aspects, human CFI is detectable in the vitreous humor at least 4 weeks after intravitreal injection of the rAAV vector. In some aspects, the level of C3b-inactivating activity in an eye of the subject is increased. In some aspects, the level of iC3b-degradation activity in an eye of the subject is increased. In certain aspects, the rAAV vector is administered as an intravitreal injection. In some aspects, the human CFI expressed from the rAAV vector is capable of degrading C3b into iC3b.

In an embodiment, a polynucleotide cassette for increasing the concentration of human CFI in a mammalian eye is provided. A polynucleotide cassette for increasing the concentration of human CFI in a mammalian eye comprises (a) an enhancer and promoter region, (b) a chimeric inton, (c) a Kozak sequence, (d) a unique coding sequence optimized for increased expression and low CpG, operatively linked to the promoter region wherein the coding sequence is a human CFI gene, (e) a human scaffold attachment region, and a polyadenylation site. In various aspects, the polynucleotide cassette is for increasing the concentration of human CFI in the vitreous humor and/or retinal extracellular space of a mammalian eye.

Methods of treating dry-AMD in a human subject comprising administering to a subject in need thereof, a therapeutically effective amount of a recombinant adeno-associated virus (rAAV) vector are provided. The rAAV vector comprises an AAV2 capsid variant and a polynucleotide cassette comprising (a) an enhancer and promoter region, (b) a chimeric intron, (c) a Kozak sequence, (d) a unique coding sequence optimized for increased expression and low CpG, operatively linked to the promoter region wherein the coding sequence is a human CFI gene, (e) a human scaffold attachment region and (f) a polyadenylation site. In various aspects, the rAAV vector is administered intravitreally. In some aspects, the therapeutically effective amount is a dosage ranging from about 1E9 to about 4E12 vector genomes (vg)/eye. In certain aspects, there is a reduction in the geographic atrophy area and/or in the rate of growth of the geographic atrophy area after administration of the rAAV vector.

An embodiment of the application provides an intravitreal dosage form comprising a recombinant adeno-associated virus (rAAV) vector at a dosage ranging from 1E9 to about 4E12 vg/eye, wherein the rAAV vector comprises a polynucleotide cassette of the application and wherein the rAAV vector comprises an AAV2 capsid variant.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 provides a schematic representation of a human Complement Factor I (hCFI) transgene expression cassette. The cassette comprises AAV2 inverted terminal repeats (ITRs) flanking the sequence. The expression cassette contains regulatory elements obtained from the human cytomegalovirus (CMV) immediate-early enhancer and promoter, an adenovirus tripartite leader sequence (TPL) followed by an enhancer element from the major late promoter (eMLP) and the intron from the mouse IgH (forming a chimeric intron), and a Kozak sequence driving expression of the codon-optimized CFI cDNA. The CFI cDNA is followed by a human scaffold attachment region (SAR) and the human growth hormone (HGH) polyadenylation site. The various elements are indicated on the schematic. The size of the vector is 4,291 bp including ITRs.

FIGS. 2A and 2B depict a characterization of different variants of human CFI cDNA developed and evaluated herein. FIG. 2A provides an illustration of the differences between wild-type CFI cDNA (solid black line, (wt_in_vector)) and five codon-optimized cDNA sequences. The codon-optimized cDNA sequences are indicated as CFI_std (standard), CFI_1.0, CFI_1.5-3, CFI_0.6, and CFI_2.0. Each vertical line represents a nucleotide change in relation to the wild-type sequence. This illustration was generated using the Geneious Prime software after alignment of the different codon-optimized cDNAs against the human wild-type CFI sequence. FIG. 2B provides an image of a Western blot analysis of five different versions of CFI under transcriptional control of the regulatory elements shown in FIG. 1. The CFI version used in the experiment is indicated at the top of each lane. The ~75 kDa band corresponds to the human Pro-CFI. The ~45 kDa band corresponds to the human CFI heavy chain. Cell culture media from untransfected cells (Untreated Cells) were used as negative controls. The values of the ladder bands (kDa) are indicated at the left of the gel image (M lane).

FIG. 3 provides an image of an alkaline gel electrophoresis result obtained from the AAV-CF1-1.0 expression cassette packaged into AAV2.7m8 (Sf9 produced) and AAV2.5T-LSV1 (HEK293 produced) capsid serotypes (samples marked with boxes were used to inject the non-human primates (NHPs) and the methods described in Examples 3 and 4 were used to produce these AAV preparations). Equal amounts (2E10 vg) of each AAV preparation were loaded into the indicated well of the gel (CFI-1.0 (LSV1) and CFI-1.0 (7m8)). One skilled in the art recognizes that 1E # notation format is equivalent to the 1×10^{#} notation format. A ladder marker was loaded into the gel well indicated with M. The values of the ladder bands (kilobases) are indicated at the left of the gel image. Sample 1 corresponds to an AAV2.7m8-CFI-1.0 preparation generated using a 60 mL culture volume of the AAV-MAX Helper-free Production system (Gibco) and purified via AVB batch binding purification. Sample 2 corresponds to an AAV2.7m8-CFI-1.0 preparation generated using a 7.2 L culture volume of the AAV-MAX Helper-free Production system (Gibco) and purified via AVB batch binding purification followed by CsCl centrifugation. Sample 3 corresponds to an AAV2.7m8-CFI-1.0 preparation generated using Sf9 cells and purified via AAVX column purification. See, for example, U.S. Pat. No. 8,945,918. A DNA band at the expected sized of 4,291 bp is present in the two lanes containing the AAV-CFI-1.0 vector.

FIGS. 4A and 4B provide images of SDS-PAGE analysis of the AAV-CFI-1.0 vector packaged into AAV2.7m8 (FIG. 4A) and AAV2.5T-LSV1 (FIG. 4B, CFI-1.0(LSV1)) capsid serotypes (samples marked with boxes were used to inject the non-human primates (NHPs) and the methods described in Examples 3 and 4 were used to produce these AAV preparations). In FIG. 4A, the AAV-CFI-1.0 lane is indicated by CFI-1.0 (7m8). Sample 3 corresponds to an AAV2.7m8-CFI-1.0 preparation generated using Sf9 cells and purified via AAVX column purification. Lanes 1-6 contain unrelated non-CFI AAV samples. In FIG. 4B, the AAV-CF-1.0 lane is indicated by CFI-1.0 (LSV1). The sizes of the ladder bands (kDa) are indicated at the left of the gel images (M lane). The VP1, VP2 and VP3 proteins are present at the expected ratios and sizes.

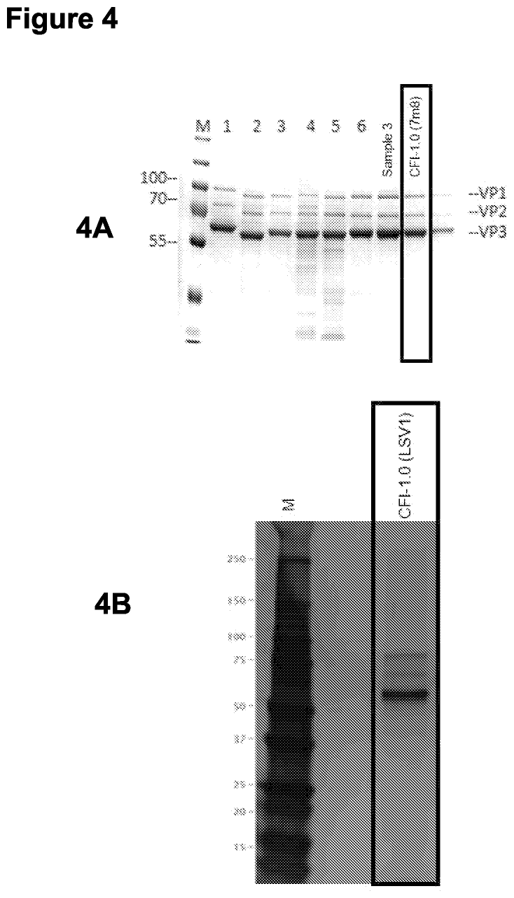

FIGS. 5A and 5B provide images of Western blot analysis of cell culture media harvested from rabbit retinal explants (FIG. 5A) and HEK293T cells (FIG. 5B) after transduction with the indicated AAV CFI-1.0 vectors packaged into the AAV2.5T-LSV1 and/or AAV2.7m8 capsid serotypes (samples marked with boxes were used to inject the non-human primates (NHPs) and the methods described in Examples 3 and 4 were used to produce these AAV preparations). FIG. 5A—Sample 2 corresponds to an AAV2.7m8-CFI-1.0 preparation generated using a 7.2 L culture volume of the AAV-MAX Helper-free Production system (Gibco) and purified via AVB batch binding purification followed by CsCl centrifugation. Sample 3 corresponds to an AAV2.7m8-CFI-1.0 preparation generated using Sf9 cells and purified via AAVX column purification. The ~75 kDa band corresponds to the human Pro-CFI. The ~45 kDa band corresponds to the human CFI heavy chain. Equal volumes were loaded in each experiment. Rabbit retinal explants were transduced in triplicate (wells labelled with A, B and C) using the following amounts of virus for the AAV2.7m8-CFI-1.0 particles, 2.5E10 vg/explant (CFI-1.0 (7m8)) and for AAV2.T-LSV1-CFI-1.0 particles, 5E10 vg/explant (CFI-1.0 (LSV1)). Samples transduced with the LSV1 AAV preparation received twice the amount of AAV compared to those transduced with the 7m8 AAV preparation. This was done as AAV2.5T-LSV1 does not transduce cells in vitro as efficiently as AAV2.7m8. Cell culture media from untransduced cells was used as a negative control (Control lanes). AAV transduction of rabbit explants has been shown to be variable, potentially due to heterogeneity of explants prepared from different areas of retina and used in each well for transduction. FIG. 5B—provides an image of Western blot analysis demonstrating similar CFI protein size and processing when produced from the CFI-1.0 vector (packaged into AAV2.7m8) and compared with human CFI present in human serum. Both HEK293T supernatant obtained from transduced cells and human serum were loaded in the gel as untreated (U) and treated with deglycosylation reagent (DG) to highlight the similar sizes and glycosylation patterns of the naïve protein and the protein produced by the CFI-1.0 vector. The human CFI protein produced from the AAV-CFI vector packaged into AAV2.7m8 is produced (size) and processed (glycosylation) similarly as the protein present in human serum. Sizes of the ladder bands are indicated at the left of the gel images (M lane).

FIG. 6 presents an image of a Western blot assay evaluating qualitatively the functionality of the CFI protein expressed and released from rabbit retinal explants after transduction with AAV CFI-1.0 vector packaged into either AAV2.7m8 or AAV2.5T-LSV1 capsid serotypes (samples marked with boxes were used to inject the non-human primates (NHPs) and the methods described in Examples 3 and 4 were used to produce these AAV preparations). Sample 2 corresponds to an AAV2.7m8-CFI-1.0 preparation generated using a 7.2 L culture volume of the AAV-MAX Helper-free Production system (Gibco) and purified via AVB batch binding purification followed by CsCl centrifugation. Sample 3 corresponds to an AAV2.7m8-CFI-1.0 preparation generated using Sf9 cells and purified via AAVX column purification. Functional activity of CFI expressed from rabbit retinal explants was evaluated using C3b cleavage assay. C3b is a cleavage substrate of CFI in the presence of CFH, a CFI cofactor. It consists of 2 subunits, alpha (116 kDa) and beta (75 kDa). When C3b is incubated with CFH and CFI, C3b is converted into iC3b. In this process the 116 kDa subunit of C3b (C3b alpha) cleaved by CFI results in formation of iC3b cleavage product, comprised of a 68 kDa polypeptide and a 43 kDa polypeptide (in bold and indicated by the arrows). To evaluate C3b cleavage, Western blot was stained with anti-C3 antibody, which binds to C3b and products of its cleavage. Cell media from rabbit explants transduced with AAV2.7m8-CFI1.0 or AAV2.T-LSV1-CFI1.0 particles (lanes A and B from CFI-1.0 (LSV1) and from CFI-1.0 (7m8) transduced explants in FIG. 5A correspond to the same lanes A and B in FIG. 6 wherein lane B had significantly less CFI protein than lane A, and subsequently less CFI activity were incubated with C3b and CFH. Lanes 2 and 12 contains a negative control with just C3b and CFH (neg control 1) and one with C3b, CFH and cell media from untransduced cells (neg control 2). Lane 3 contains a positive control with purified C3b and CFH proteins and CFI protein (pos control lane). Lanes 6 and 7 show products of reactions containing C3b, CFH and cell media from rabbit explants transduced with AAV2.T-LSV1-CFI-1.0 particles. Lanes 10 and 11 show products of reactions C3b, CFH and cell media from rabbit explants transduced with AAV2.7m8-CFI1.0 particles. The iC3b 43 kDa polypeptide is present in all lanes containing cell media from rabbit explants transduced with AAV-CFI-1.0 package into either AAV2.7m8 or AAV2.5T-LSV1 capsid serotypes. The iC3b 68 kDa polypeptide is visible in lanes 5 and 9. Lanes B had significantly less CFI protein than lane A (as shown in FIG. 5A) subsequently less CFI activity was detected in the lanes B of FIG. 6.

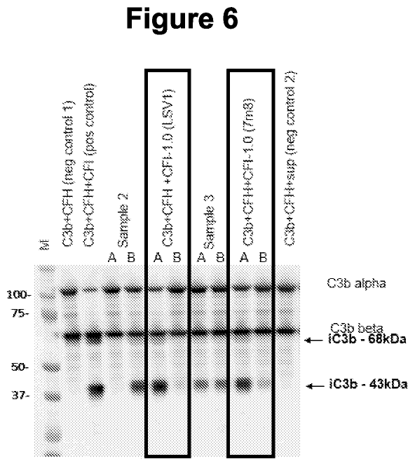

FIG. 7A provides a schematic of a non-human primate (NHP) study to evaluate human CFI expression in the vitreous humors (VH) after intravitreal (IVT) injection of AAV-CFI-1.0 vector packaged into either the AAV2.7m8 or AAV2.5T-LSV1 capsid serotypes (Methods described in Examples 3 and 4 were used to produce these AAV preparations). Two AAV doses were tested (1E11 vg/eye or 3E10 vg/eye). Three animals were injected with each AAV dose, and three animals injected with vehicle only. FIG. 7B outlines the analyses performed and the collection time points. Electroretinogram (ERG), Ocular examination (OE)/Intraocular Pressure (IOP) and Ocular Coherence Tomography (OCT)/fundus analyses were performed prior to commencement of the study. ERG was performed 21 days before study start, OE/IOP was performed 16 days before study start and OCT/fundus was performed 14 days before study start. IVT injection occurred on day 1 of the study. Vitreous humors (VH) and aqueous humors (AH) were collected at Day 28, Day 62 and Day 88 post AAV injection. In some studies, VH, AH and/or tissue are evaluated for CFI DNA or RNA at Day 88 post injection. The study was terminated at Day 88 and final samples collected.

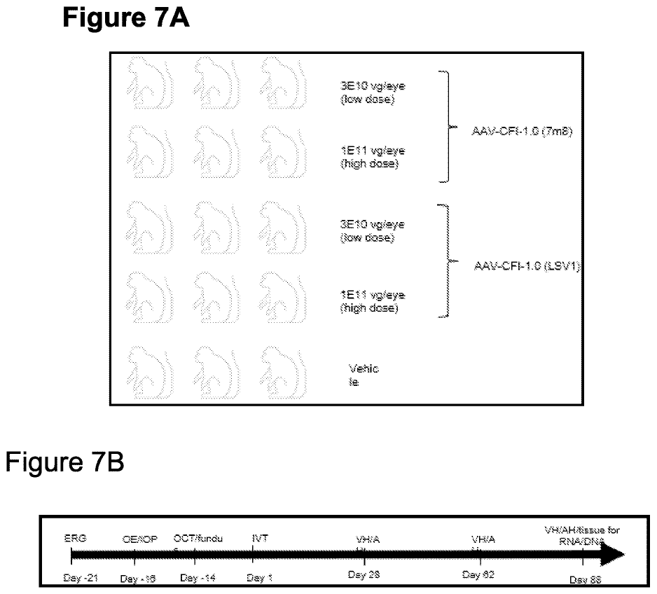

FIG. 8A provides a table summarizing the Vitreous Humors (VH) collection strategy. Oculus dexter (OD), oculus sinister (OS), oculus uterque (OU). The * denotes the two eyes from which collection was not enough VH from OD on Day 28. Then OS was collected from the two animals. FIG. 8B depicts the peak expression values observed during the entire study for each eye of the NHPs injected IVT with the AAV-CFI-1.0 vector packaged into either the AAV2.7m8 or AAV2.5T-LSV1 capsid serotypes (Methods described in Examples 3 and 4 were used to produce these AAV preparations). The VH from each eye was analyzed with a CFI Luminex assay. Human CFI (hCFI) levels in ng/mL are indicated on the y-axis and the doses in vg/eye are indicated in the x-axis of the graph. The different capsid serotypes used are indicated on the brackets located above the sample data. Horizontal lines represent mean values (n=6 for animals injected with AAV2.7m8) and n=5 for animals injected with AAV2.5T-LSV1). BLOQ indicates a value below the limit of quantification. The mean values were calculated only with samples with quantifiable values, although the BLOQ samples are indicated in the graph (denoted with *). Eyes from animals injected with vehicle only (vehicle) exhibited no detectable hCFI (BLOQ) (data not shown). The mean levels of hCFI for each group are: 2464 ng/mL for the 3E10 vg/eye group of AAV2.7m8-CFI-1.0 injected NHP, 2313 ng/mL for the 1E11 vg/eye group of AAV2.7m8-CFI-1.0 injected NHP, 575 ng/mL for the 3E10 vg/eye group of AAV2.5T-LSV1-CFI-1.0 injected NHP, and 557 ng/mL for the 1E11 vg/eye group of AAV2.5T-LSV1-CFI-1.0 injected NHP. The highest values of hCFI were obtained in the 3E10 vg/eye group of AAV2.7m8-CFI-1.0 injected NHPs, with values reaching up to 6666 ng/mL. At days 62 and 88, some animals treated with either vector and dose had hCFI levels that were BLOQ. The same animals had high levels of antibodies generated against human CFI, thus this could be a result of immune response in NHPs against human protein. Vitreous humor from NHP subject #2202 was collected at Day 79, as this animal had to be euthanized on Day 79, which was unrelated to the test article.

FIG. 9 provides a table summarizing the clinical scores of vitreous cells obtained from all animals before injections and at days 12, 27, 37, 52 and 79 post injection in the left (OS) and right (OD) eyes of the subject NHP's.

FIG. 10 provides graphs summarizing the spatial biodistribution data (vector genomes (vgs)) from different systemic and ocular tissues from non-human primates (NHPs) after intravitreal (IVT) injection of AAV-CFI-1.0 vector packaged into either the AAV2.7m8 or AAV2.5T-LSV1 capsid serotypes. Animals were dosed with 3E10 (top) and 1E11 vg/eye (bottom). Tissues were collected on Day 88 post-dose. (R) and (L) denote that the right or left part of the tissues were analyzed, respectively. LN denotes lymph node; LGN denotes lateral geniculate nucleus. Horizontal bars correspond to geometric means.

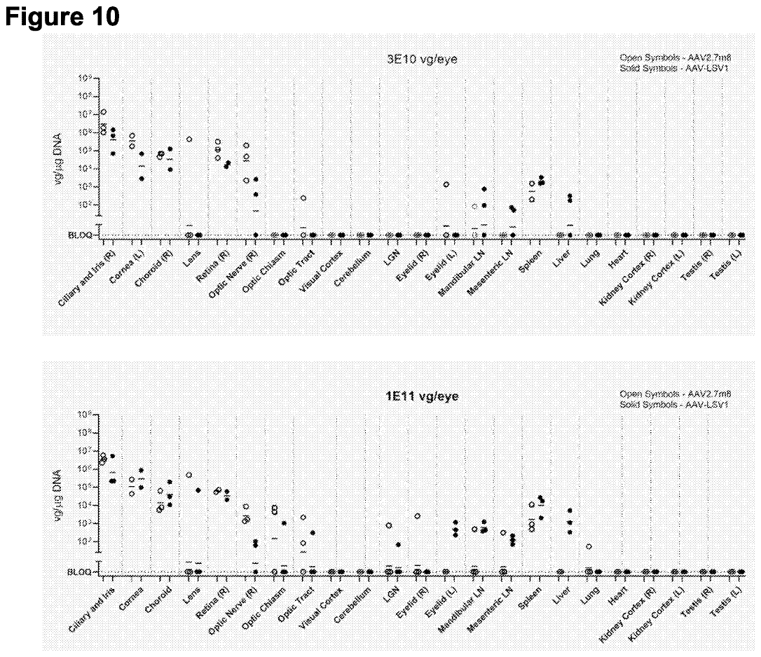

FIG. 11 provides a graph summarizing the AAV vector genome (vg) clearance data from serum obtained at different time points (Pretreatment, Day 1 (8 hours), Days 2, 3, 6, 14, 21, 28, 43, and 56) from NHPs after intravitreal (IVT) injection of AAV-CFI-1.0 vector packaged into either the AAV2.7m8 or AAV2.5T-LSV1 capsid serotypes. Animals were dosed with 3E10 (open symbols) and 1E11 vg/eye (black symbols). The time of the whole blood collection is indicated on the x-axis.

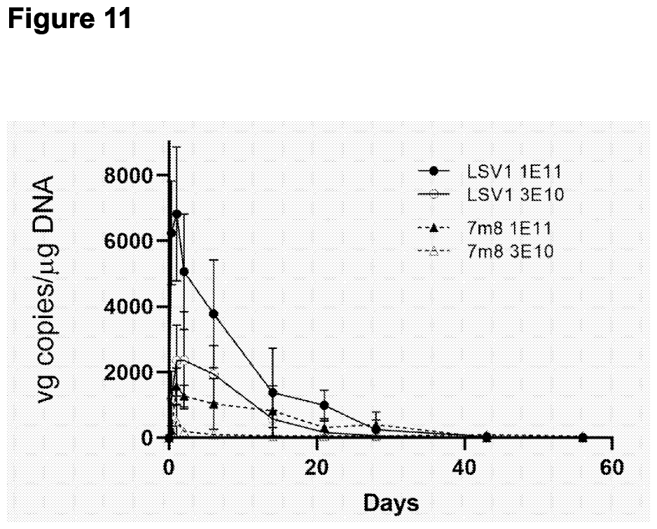

FIG. 12 presents the human CFI protein levels obtained from ocular tissues/compartments. Samples were obtained from non-human primates (NHPs) after intravitreal (IVT) injection of AAV-CFI-1.0 vector packaged into either the AAV2.7m8 (top graphs) or AAV2.5T-LSV1 (bottom graphs) capsid serotypes. Animals were dosed with 3E10 (left graphs) and 1E11 vg/eye (right graphs). Tissues were collected on Day 88 post-dose. Tissue lysates were prepared using choroid, Iris plus Ciliary body (ICB) and Retina. FIG. 12A depicts the data from tissues lysates using graphs with the human CFI levels obtained using the Luminex assay. Human CFI levels are represented in ng/g for tissue lysates, and as ng/mL for vitreous humor (VH). Solid bars correspond to geometric means. FIG. 12B depicts a table containing the same information represented in FIG. 12A but using a different representation system to describe the human CFI levels in each sample: −: BLOQ, +: 1-100 ng, ++: 101-600 ng, +++: >601 ng.

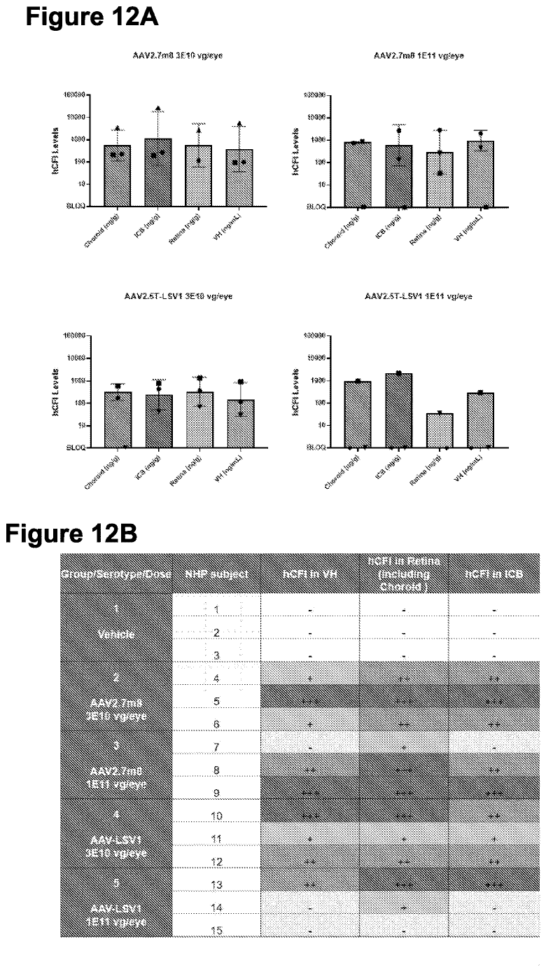

FIG. 13 provides a visual representation of AAV vector genome biodistribution in eyes obtained from non-human primates (NHPs) after intravitreal (IVT) injection of AAV-CFI-1.0 vector packaged into either the AAV2.7m8 or AAV2.5T-LSV1 capsid serotypes. Animals were dosed with 3E10 (top) and 1E11 vg/eye (bottom). Arrowheads point to some of AAV vector DNA—positive cells and extracellular particles in photomicrographs of samples. For vectors packaged in AAV2.7m8: In the Macula (panels A, E), intracellular vector DNA is detected in retinal ganglion cells and inner nuclear layer cells. In the Mid-periphery (panels B, F), no intracellular vector DNA is detected; vector DNA is detected associated with inner limiting membrane. In the Far-Peripheral retina (panels C, G), intracellular vector DNA is detected in cells within the retinal ganglion and inner nuclear cell layers. In the Ciliary body (panels D, H), intracellular vector DNA is detected in some nonpigmented epithelial cells of ciliary processes. For vectors packaged in AAV2.5T-LSV1: in the Macula (panels I, M), intracellular DNA is detected in retinal ganglion cells and inner nuclear layer cells. In the Mid-periphery (panels J, N), intracellular DNA is detected in retinal ganglion cells and inner nuclear layer cells. In the Far-periphery (panels K, O), trace levels of intracellular DNA is detected in retinal ganglion cells and inner nuclear layer cells. In the Ciliary body (panels L, P), intracellular DNA can be detected in ciliary muscle and epithelial cells surrounding Schlemm's canal.

FIG. 14 provides a visual representation of AAV-CFI transgene mRNA biodistribution in eyes obtained from non-human primates (NHPs) after intravitreal (IVT) injection of AAV-CFI-1.0 vector packaged into either the AAV2.7m8 or AAV2.5T-LSV1 capsid serotypes. Animals were dosed with 3E10 (top) and 1E11 vg/eye (bottom). Arrowheads point to some positive cells in photomicrographs of samples. In general, for both AAV2.7m8 and AAV2.5T-LSV1, mRNA is mainly produced in the macula (by retinal ganglion cells, inner nuclear cells, and photoreceptors), retina (far-periphery) and ciliary process. More specifically, for vectors packaged in AAV2.7m8: In the Macula (panels A, D), vector mRNA is detected in retinal ganglion cells, inner nuclear layer cells, and sparingly in photoreceptors. In the Peripheral retina (panels B, E), vector mRNA is detected in retinal ganglion cells, inner nuclear layer cells, and sparingly in photoreceptors. In the Ciliary body (panels C, F), vector mRNA is detected in some nonpigmented epithelial cells of ciliary processes. For vectors packaged in AAV2.5T-LSV1: in the Macula (panels G, K), vector mRNA is detected in retinal ganglion cells, and inner nuclear layer cells. In the Mid-periphery (panels H, L), vector mRNA is detected in retinal ganglion cells and inner nuclear cells. In the Far-periphery (panels I, M), vector mRNA is detected in retinal ganglion cells and inner nuclear cells. In the Ciliary body (panels J, N) vector mRNA can be detected in ciliary muscle, ciliary process non-pigmented epithelial cells, and epithelial cells surrounding Schlemm's canal.

FIG. 15 provides a summary of the levels of anti-drug antibody (ADA) developed against the human CFI protein detected in serum from non-human primates (NHPs) injected intravitreal (IVT) injection with the AAV-CFI-1.0 vector packaged into either the AAV2.7m8 or AAV2.5T-LSV1 capsid serotypes. Animals were dosed with 3E10 and 1E11 vg/eye. ADA formation was anticipated given the amino acid differences between the human and NHP CFI proteins. Values are representative of electrochemiluminescence (ECL) signal. FIG. 15A presents the raw ECL values for serum collected at Baseline, Day 56 and Day 88 post-injection of AAV vectors. N/A represents a sample that could not be collected for analysis. FIG. 15B represents the same data set, but utilizing a different score system based on the ECL values: −: <199, +: 200-10000, ++: 10001-100000, +++: 100000-1000000.

FIG. 16 provides graphs summarizing the spatial biodistribution data (mRNA produced from AAV vector) from different systemic and ocular tissues from non-human primates (NHPs) after intravitreal (IVT) injection of AAV-CFI-1.0 vector packaged into either the AAV2.7m8 or AAV2.5T-LSV1 capsid serotypes. Animals were dosed with 3E10 (top) and lxii vg/eye (bottom). Tissues were collected on Day 88 post-dose. (R) and (L) denote that the right or left part of the tissues were analyzed, respectively. LGN denotes lateral geniculate nucleus. Horizontal bars correspond to geometric means.

### DETAILED DESCRIPTION

The following passages describe different aspects of the invention in greater detail. Each aspect, embodiment, or feature of the invention may be combined with any other aspect, embodiment, or feature the invention unless clearly indicated to the contrary. All patents and publications, referred to herein are expressly incorporated by reference in their entirety.

Unless defined otherwise, all technical and scientific terms used herein have the meaning commonly understood by a person skilled in the art to which this invention belongs.

Numeric ranges are inclusive of the numbers defining the range. The term about is used herein to mean plus or minus ten percent (10%) of a value. For example, “about 100” refers to any number between 90 and 110.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

Unless otherwise defined, all technical terms used herein have the same meaning as commonly understood by one of ordinary skill in the art.

One skilled in the art recognizes that 1E # notation format is equivalent to the 1×10 # notation format.

The terminology used herein is for the purpose of describing particular examples only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “comprising” as used herein is synonymous with “including” or “containing,” and is inclusive or open-ended.

By “consisting essentially of”, is intended a limitation of the scope of the, for example, composition, method, kit, etc., described to the specified materials that do not materially affect the basic and novel characteristic(s) of the, for example, composition, method, kit, etc. For example, an expression cassette “consisting essentially of” a coding sequence encoding a polynucleotide operably linked to a promoter and a polyadenylation sequence may include additional sequences, e.g., linker sequences so long as they do not materially affect the transcription or translation of the coding sequence. As another example, a variant or mutant polypeptide “consisting essentially of” a recited sequence has the amino acid sequence of the recited sequence plus or minus about 10 amino acid residues at the boundaries of the sequence based upon the full length naïve polypeptide from which it was derived, e.g., 10, 9, 8, 7, 6, 5, 4, 3, 2 or 1 residue less than the recited bounding amino acid residue or 1, 2, 3, 4, 5, 6, 7, 8, 9 or 10 residues more than the recited bounding amino acid residue.

Any reference to “or” herein is intended to encompass “and/or” unless otherwise stated. As used herein, the term “about” a number refers to that number plus or minus 10% of that number. The term “about” a range refers to that range minus 10% of its lowest value and plus 10% of its greatest value. Reference to “about” a value or parameter herein includes (and describes) embodiments that are directed to that value or parameter per se.

The term “subject”, “patient”, or “individual” refers to a mammal including but not limited to, primates, such as humans and non-human primates, e.g., African green monkeys and rhesus monkeys, mammalian sport animals, mammalian farm animals, mammalian pets and rodents. In some embodiments, the subject is a human.

The terms “treat,” “treating”, “treatment,” “ameliorate” or “ameliorating” and other grammatical equivalents as used herein, refer to alleviating, abating or ameliorating dry age-related macular degeneration (dry-AMD) disease or disorder, or symptoms of dry-AMD disease or disorder, preventing additional symptoms of the dry-AMD disease or disorder, ameliorating or preventing the underlying causes of symptoms, inhibiting dry-AMD disease or disorder, e.g., arresting the development of dry-AMD disease or disorder, relieving dry-AMD disease or disorder, causing regression of dry-AMD disease or disorder, or stopping the symptoms of dry-AMD disease or disorder, and are intended to include prophylaxis and prevention of wet-AMD. The terms further include achieving a therapeutic benefit and/or a prophylactic benefit. The term “therapeutic benefit” refers to eradication or amelioration of dry-AMD disease or disorder being treated. Also, a therapeutic benefit is achieved with the eradication or amelioration of one or more of the physiological symptoms associated with dry-AMD disease or disorder such that an improvement is observed in the subject, notwithstanding that, in some embodiments, the subject is still afflicted with dry-AMD disease or disorder. For prophylactic benefit, the pharmaceutical compositions are administered to a subject at risk of developing dry-AMD disease or disorder, or to a subject reporting one or more of the physiological symptoms of dry-AMD disease or disorder, even if a diagnosis of the disease or disorder has not been made.

Signs and symptoms of dry-AMD include, but are not limited to, endothelial cell proliferation, retinal pigment epithelium (RPE) atrophy,

One skilled in the treatment of diseases and disorders of the eye would be familiar with the structure of the mammalian eye, particularly the human eye. The “retina” is a multi-layered membrane that lines the inner posterior chamber of the eye and senses an image of the visual world which is communicated to the brain via the optic nerve. In order from the inside to the outside of the eye, the retina comprises the layers of the neurosensory retina and retinal pigment epithelium (RPE), with the choroid lying outside the retinal pigment epithelium. The neurosensory retina harbors the photoreceptor cells that directly sense light.

The neurosensory retina comprises the following layers: internal limiting membrane (ILM), nerve fiber layer, ganglion cell layer, inner plexiform layer, inner nuclear layer, outer plexiform layer, outer nuclear layer (nuclei of the photoreceptors); external limiting membrane (ELM) and photoreceptors (inner and outer segments of the rods and cones). Photoreceptor cells are specialized neurons located in the retina that convert light into biological signals. Photoreceptor cells comprise rod cells and cone cells which are distributed differently across the retina. Rod cells are distributed mainly across the outer parts of the retina. Rod cells are highly sensitive and provide for vision at low light levels. A normal human retina contains about 125 million rod cells, on average. Cone cells are found across the retina but are particularly highly concentrated in the fovea, a pit in the neurosensory retina that is responsible for central high resolution vision. Cone cells are less sensitive than rod cells. There are on average about 6-7 million cone cells in a normal human retina.

The retinal pigment epithelium (RPE) is a pigmented layer of cells located immediately to the outside of the neurosensory retina. The RPE performs a number of functions including, but not limited to, transport of nutrients and other substances to the photoreceptor cells and absorption of scattered light to improve vision.

The choroid is the vascular layer situated between the RPE and the outer sclera of the eye. The vasculature of the choroid enables provision of oxygen and nutrients to the retina.

The term “retinal extracellular space” is intended to encompass the space in the retina outside of the plasma membrane of neurons and glia. The “vitreous humor”, “vitreous fluid” and “vitreous body” is a mostly clear gel that fills the space between the lens and the retina of the eyeball in humans and other mammals composed mainly of water and a fibrillar meshwork of collagenous extracellular matrix associated with hyaluronic acid. The vitreous humor supports eye structures and helps maintain transparency of the media.

By “aqueous humor” and “aqueous fluid” is intended the clear liquid inside the front part of the eye between the lens and the cornea. It nourishes the eye and keeps it inflated. The eye constantly produces a small amount of aqueous humor.

The terms “administer,” “administering”, “administration,” and the like, as used herein, can refer to the methods that are used to enable delivery of therapeutics or pharmaceutical compositions to the desired site of biological action. These methods include intravitreal or subretinal injection to an eye.

The terms “effective amount”, “therapeutically effective amount” or “pharmaceutically effective amount” as used herein, can refer to a sufficient amount of at least one pharmaceutical composition or compound being administered which will relieve to some extent one or more signs or symptoms of the ocular disease, ocular disorder or ocular condition being treated. An “effective amount”, “therapeutically effective amount” or “pharmaceutically effective amount” of a pharmaceutical composition may be administered to a subject in need thereof as a unit dose (as described in further detail elsewhere herein). The subject may be a human or non-human mammal.

The term “pharmaceutically acceptable” as used herein, can refer to a material, such as a carrier or diluent, which does not abrogate the biological activity or properties of a compound disclosed herein, and is relatively nontoxic (i.e., when the material is administered to an individual it does not cause undesirable biological effects nor does it interact in a deleterious manner with any of the components of the composition in which it is contained).

The term “pharmaceutical composition,” or simply “composition” as used herein, can refer to a biologically active compound, optionally mixed with at least one pharmaceutically acceptable chemical component, such as, though not limited to carriers, stabilizers, diluents, dispersing agents, suspending agents, thickening agents, excipients and the like.

A “vector” as used herein refers to a macromolecule or association of macromolecules that comprises or associates with a polynucleotide and which can be used to mediate delivery of the polynucleotide to a cell. Illustrative vectors include, but are not limited to, plasmids, viral vectors (i.e., adeno-associated viruses), liposomes and other gene delivery vehicles.

An “AAV vector” or “rAAV vector” as used herein refers to an adeno-associated virus (AAV) vector or a recombinant AAV (rAAV) vector comprising a polynucleotide sequence not of AAV origin (e.g., a polynucleotide heterologous to AAV such as a nucleic acid sequence that encodes a therapeutic transgene, e.g., human complement factor inhibitor (CFI) for transduction into a target cell or to a target tissue. In general, the heterologous polynucleotide is flanked generally by two AAV inverted terminal repeat sequences (ITRs). The term rAAV vector encompasses both rAAV vector particles and rAAV vector plasmids. A rAAV vector may be either single-stranded (ssAAV) or self-complementary (scAAV).

An “AAV virus” or “AAV viral particle” or “rAAV vector particle” or “rAAV particle” refers to a viral particle comprising at least one AAV capsid protein and a polynucleotide rAAV vector. In some cases, the at least one AAV capsid protein is from a wild type AAV or is a variant AAV capsid protein. By “variant AAV capsid protein” is intended that the AAV capsid protein comprises at least one amino acid difference (e.g., amino acid substitution, amino acid insertion, amino acid deletion) relative to a corresponding parental AAV capsid protein. The variant capsid protein may confer increased infectivity of a retinal cell as compared to the infectivity of a retinal cell by an AAV virion comprising an amino acid sequence present in a naturally occurring AAV capsid protein. Variant AAV capsid proteins may include, but are not limited to, an AAV capsid protein with an insertion, an insertion of the 7m8 amino sequence, an R100 insertion, a 7m8 like insertion, an LSV1 sequence replacement and any other engineered capsid protein generated by other strategies (e.g., DNA shuffling, directed evolution, peptide insertion, ancestral reconstruction, among others). The LSV1 replacement sequence and the 7m8 insertion sequence are known in the art (see, for example, U.S. Pat. Nos. 9,193,956; 9,233,133; U.S. Pub. No. US2021/0040501; and PCT/US2020/029895). If the particle comprises a heterologous polynucleotide (e.g., a polynucleotide other than a wild-type AAV genome such as a transgene to be delivered to a target cell or target tissue), it is referred to as a “rAAV particle”, “rAAV vector particle” or a “rAAV vector”. Thus, production of rAAV particles necessarily includes production of a rAAV vector, as such a vector contained within a rAAV particle. In general, the heterologous polynucleotide is flanked by AAV inverted terminal repeat sequences (ITRs). A heterologous polynucleotide may comprise a polynucleotide cassette. A polynucleotide cassette of the present application can be packaged in a variant AAV particle to promote delivery of the cassette to a cell type of interest such as, but not limited to a retinal cell, in a target tissue.

The term “packaging” as used herein can refer to a series of intracellular events that can result in the assembly and encapsidation of a rAAV particle.

AAV “rep” and “cap” genes refer to polynucleotide sequences encoding replication and encapsidation proteins of adeno-associated virus. AAV rep and cap are referred to herein as AAV “packaging genes.”

A “non-naturally occurring” polynucleotide cassette is one that is not found in nature.

By “polynucleotide cassette” is meant a polynucleotide sequence comprising two or more functional polynucleotide sequences, e.g., regulatory elements, translation initiation sequences, coding sequences, termination sequences, etc. typically in operable linkage to at least one other functional polynucleotide sequence in the polynucleotide cassette. Generally, a subject polynucleotide cassette is composed of DNA. Likewise, a “polynucleotide cassette for enhanced expression of a transgene in the vitreous humor of a mammalian eye” is intended to mean a combination of two or more polynucleotide sequences, e.g., promoter, enhancer, 5′UTR, translation initiation sequence, coding sequence, termination sequences, etc that promote enhanced expression of a transgene in the vitreous humor of a mammalian eye.

For example, in some embodiments, the polynucleotide cassette comprises in 5′ to 3′ order: (a) optionally a first enhancer region; (b) a promoter region, wherein the promoter region is specific for eukaryotic cells; (c) a coding sequence encoding a polynucleotide gene product; (d) a second enhancer region; and (e) a polyadenylation site. In still other embodiments, the polynucleotide cassette further comprises a ‘5′untranslated region’ (5′UTR) upstream of the coding sequence. In yet other embodiments, the polynucleotide cassette further comprises an intron region downstream of the promoter and upstream of the coding sequence. In other embodiments, the polynucleotide cassette further comprises the RNA export signal downstream of the second enhancer and upstream of the polyadenylation site. In regard to the polynucleotide cassettes disclosed herein, the coding sequence is understood to be operably linked to the expression control sequences in the cassette. For example, the coding sequence is operably linked to the promoter region, enhancer region(s) and the polyadenylation site.

Without being limited by mechanism, in some embodiments, the polynucleotide cassettes of the present application provide enhanced expression of the hCFI transgene by a retinal cell and enhanced secretion of hCFI within the retinal extracellular space, in the vitreous humor of the mammalian eye or both within the retinal cell extracellular space and in the vitreous humor of the mammalian eye. In certain embodiments, the arrangement of the two or more functional polynucleotide sequences within the polynucleotide cassettes of the present disclosure provide for enhanced expression of a CFI transgene in the vitreous humor of a mammalian eye. By “enhanced” it is intended that expression of the CFI transgene is increased, augmented, greater than, or stronger in mammalian eyes carrying the polynucleotide cassettes of the present disclosure relative to mammalian eyes that lack a polynucleotide cassette of the present application. In some embodiments, enhanced expression of the CFI transgene occurs in ocular cells near the vitreous humor. It is recognized that enhanced expression of the CFI transgene may specifically occur in one or more ocular cell types or may be limited to one or more ocular cell types. In an embodiment the CFI transgene encodes a protein that is secreted by a cell into the aqueous environment surrounding a cell, this may result in an increased concentration of the CFI transgene in the vitreous humor of a mammalian eye.

In preferred embodiments, the polynucleotide expression cassette promotes expression and secretion (or a higher level of expression and secretion as compared to a reference cassette) of the transgene into the vitreous humor of a mammalian eye in vitro or in vivo, or locally, within the retinal extracellular space. Examples of cell types include, but are not limited to, HeLa cells, HEK293 cells, ARPE-19 cells (a human retinal pigment epithelial cell line), retinal ganglion cells, amacrine cells, horizontal cells, bipolar cells, photoreceptor cells, cone cells, rod cells, Muller glial cells and retinal pigmented epithelium. In another embodiment, enhanced expression is observed in vitreous humor of retinal tissue explants.

As used herein, the terms “gene” and “coding sequence” refer to a nucleotide sequence that encodes a gene product in vitro or in vivo. The term “transgene” refers to a coding sequence or gene that is delivered into a cell by a vector. The coding sequence or gene may encode a peptide or polypeptide molecule. As used herein, the term “gene product” refers to the desired expression product of a polynucleotide sequence such as a peptide or protein. As used herein, the terms “polypeptide” and “protein” refer to polymers of amino acids of any length. The term “peptide” refers to a polymer of amino acids of about 50 or fewer amino acids. The terms also encompass an amino acid polymer that has been modified, as by for example, disulfide bond formation, glycosylation, lipidation or phosphorylation. In some instances, a polypeptide may have a length greater than 50 amino acids.

The polynucleotide cassettes of the present disclosure typically comprise a promoter region. A “promoter” as used herein encompasses a DNA sequence that directs the binding of RNA polymerase and thereby promotes RNA synthesis. Promoters and corresponding protein or polypeptide expression may be ubiquitous, meaning strongly active in a wide range of cells, tissues and species, or cell-type specific, tissue-specific or species specific. Promoters may be “constitutive”, meaning continuously active or “inducible” meaning the promoter can be activated or deactivated by the presence or absence of biotic or abiotic factors. In certain embodiments, the promoter region promotes expression of the coding sequence in mammalian cells. Suitable examples include the actin, chicken β-actin (CBA), cytomegalovirus (CMV), CMV immediate enhancer/β-actin (CAG), elongation factor 1 alpha (EF1a), and glyceraldehyde 3-phosphate dehydrogenase (GAPDH) promoters. A promoter may show retinal specific expression. Thus, expression from a promoter may be retinal-cell specific, for example primarily occurring in cells of the neurosensory retina and retinal pigment epithelium. Examples of retina-specific promoters include, but are not limited to, rhodopsin kinase for rods and cones, PR2.1 for cones only, RPE65 and VMD2 for the RPE. See Allocca et al (2007) J. Virol. 81:11372-80, Mancuso et al (2009) Nature 461:784-787, Bainbridge et al (2008) N. Eng. J. Med 358:2231-2239, and Esumi et al (2004) J. Biol. Chem. 279:19064-73.

In some embodiments, the polynucleotide cassette comprises one or more enhancers. Enhancers are nucleic acid elements that enhance transcription. Enhancer sequences may or may not be contiguous with the promoter sequence. Enhancer sequences influence promoter-dependent gene expression and may be located in the 5′ or 3′ regions of the native gene. An “enhancer” as used herein encompasses a cis-acting element that stimulates or inhibits transcription of adjacent genes or coding sequences. An enhancer that inhibits transcription also is termed a “silencer”. Enhancers can function (i.e., be associated with a coding sequence in either orientation, over distances of up to several kilobase (kb) pairs from the coding sequence and from a position downstream of a transcribed region. In some embodiments, the polynucleotide cassette comprises a first enhancer upstream of the coding sequence and a second enhancer downstream of the coding sequence. Exemplary suitable enhancers include, but are not limited to, EF1a, CMV, CAG, the full EES or a portion of the EES, such as the 410-564 EES or 511-810 EES. In certain embodiments, the upstream enhancer includes but is not limited to EF1a and CMV. In certain embodiments, the downstream enhancer includes, but is not limited to the full expression enhancer sequence (full EES), 410-564 EES or 511-810 EES. Ocular gene enhancers known in the art may be used in the methods of the application. The terms “ocular gene enhancer” and “ocular enhancer” are used interchangeably herein. Ocular gene enhancers include, but are not limited to, the CRX/OTX2 enhancer, the NRF1 enhancer, the MAF/NRL enhancer, the MEF2 enhancer, the RORA/RORB enhancer, the AP-1 enhancer, the MITF enhancer, and the TEAD enhancer. See, for example, Cherry et al 2020 PNAS 117(16):9001-9012. It is recognized that enhancers for expression in the macula may include, but are not limited to, CRX/OTX2, NRF1, MAF/NRL and MEF2. It is recognized that enhancers for expression in the retina may include, but are not limited to, CRX/OTX2, MAF/NRL, RORA/RORB and MEF2. It is recognized that enhancers for expression in the RPE and the choroid may include, but are not limited to, AP-1, MITF, TEAD and OTX/CRX.

In some embodiments, the polynucleotide cassette comprises a sequence encoding a 5′ UTR, i.e., a polynucleotide sequence encoding an untranslated region 5′ to the coding sequence. In some embodiments, the 5′ UTR does not contain the polynucleotide ATG. Exemplary suitable 5′UTR sequences include, but are not limited to, sequences selected from i) the tripartite leader sequence (TPL) from adenovirus (Logan et al (1984) Proc. Natl Acad Sci. 81:3655-3659); ii) the enhancer element sequence from the adenovirus major late promoter (eMLP) (Durocher et al (2002) Nucl. Acids Res. 30(2):e9); iii) UTR1; and iv) UTR2. In a preferred embodiment, the 5′UTR comprises in a 5′ to 3′ order, a TPL and an eMLP sequence.

In some embodiments the subject polynucleotide cassette further comprises an intron comprising a splice donor/acceptor region. In some embodiments, the intron is located downstream of the promoter region and is located upstream of the translation initiation sequence of the gene. Introns are DNA polynucleotides that are transcribed into RNA and removed during mRNA processing through intron splicing. Polynucleotide cassettes containing introns generally have higher expression than those without introns. Introns can stimulation expression between 2- and 500-fold (Buchman & Berg (1988) Mol. Cell Biol. 8(10):4395). Efficiently spliced introns contain a pre-splice donor, branch point and Py rich region. Although introns are known to generally increase the level of gene expression, the actual increase (if any) of any given cDNA is empirical and must be determined. See US Patent Pub. No: US2021/0040501. Exemplary intron sequences include, but are not limited to, sequences from actin, chicken 3-actin (CBA), rabbit globin intron, elongation factor 1 alpha (EF1a), enhancer element from the adenovirus major late promoter (eMLP) and CMVc. Other intron sequences include, but are not limited to, chimeric sequences of HBB-IGG (comprising the 5′ donor site from the first intron of the human β-globin gene and the branch and 3′ acceptor site from the intron of an immunoglobulin gene heavy chain variable region), chicken 3-actin (CBA), rabbit globin intron and exon 3 of the β-globin gene, or modified versions of the introns above, generated after deletion of different nucleotide sequences within the intron, mammalian introns, MAT2A, rpL32, and those described in Hube et al (2015) Int J. Mol. Sci 16(3):4429-4452.

The transgene product may act intrinsically in a mammalian cell, it may act extrinsically (i.e., it may be secreted), or the transgene product act both intrinsically and extrinsically.

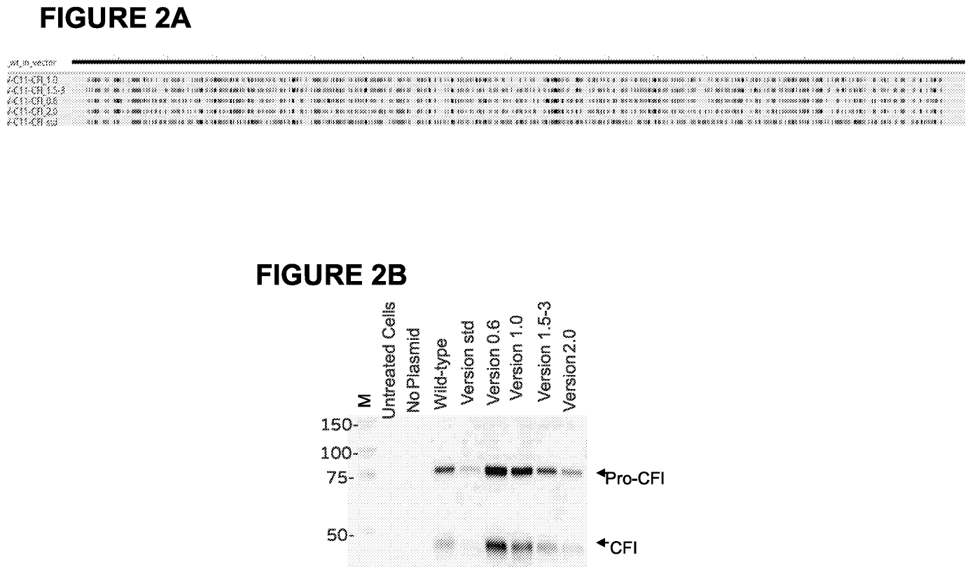

In various embodiments, the human CFI transgene coding sequence may be modified, or “codon optimized” to enhance expression by replacing infrequently represented codons with more frequently represented codons. The coding sequence is the portion of the mRNA sequence that encodes the amino acids for translation. Codon optimization is an unpredictable art. While translation may be improved via “codon optimization” for a particular species, where the coding sequence is altered to encode the same amino acid sequence by utilizing codons that are highly represent and/or utilized by highly expressed human proteins (Cid-Arregui et al (2003) J. Virol 77:4928), codon optimization is not always beneficial (Driesmann et al (2022) Gene Therapy 28:265-276). Codon optimized versions of human CFI resulted in reduced CFI secretion compared to the non-optimized CFI sequence (wild-type) (Driesmann et al (2022) Gene Therapy 28:265-276). Importantly, at least two of the novel codon-optimized forms of human CFI described herein (hCFI cDNA version 0.6 and 1.0) are expressed and secreted more efficiently than the non-optimized CFI sequence (wild-type). CFI 1.0, CFI 1.5-3, CFI 0.6, and CFI 2.0 are codon-optimized variants of CFI; the variants are described more fully in FIG. 2A.

In some embodiments, the coding sequence of the transgene encodes a polypeptide having at least 95% identity to a polypeptide encoded by the wild-type human CFI; for example at least 95% sequence identity, at least 96% sequence identity, at least 97% sequence identity, at least 98% sequence identity, at least 99% sequence identity to human CFI and wherein at least one codon of the coding sequence has a higher tRNA frequency in humans than the corresponding codon in the wild-type CFI sequence. In some embodiments, the coding sequence of the transgene encodes a polypeptide having at least 99% sequence identity to a polypeptide encoded by the wild-type human CFI and the coding sequence has at least 95% sequence identity, at least 96% sequence identity, at least 97% sequence identity, at least 98% sequence identity, or at least 99% sequence identity to the wild-type human CFI coding sequence. In some embodiments the coding sequence of the transgene is selected from the group of coding sequences comprising SEQ ID NO:2, SEQ ID NO:3, SEQ ID NO:4, SEQ ID NO:5 and SEQ ID NO:6.

As used herein, the terms “sequence identity”, “percent identity” and “percent sequence identity” refer to the degree of identity between two or more polynucleotides when aligned using a nucleotide sequence alignment program; or between two or more polypeptide sequences when aligned using an amino acid sequence alignment program. Similarly, the terms “identical” and percent “identity” when used herein in the context of two or more nucleotide or amino acid sequences refers to two sequences that are the same or have a specified percentage of amino acid residues or nucleotides when compared and aligned for maximum correspondence, for example as measured using a sequence comparison algorithm, e.g., the Smith-Waterman algorithm, etc. or by visual inspection. The percent identity between amino acid sequences may be determined using by, for example, the Needleman and Wunsch (1970, J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6 or 4 and a length weight of 1, 2, 3, 4, 5, or 6. As another example, the percent identity between two nucleotide sequences may be determined using the GAP program in the GCG software package, using a NWSgapdna.CMP matrix and a gap weight of 40, 50, 60, 70, or 80 and length weight of 1, 2, 3, 4, 5 or 6. A particularly preferred set of parameters (and the one that should be used unless otherwise specified) are a Blossum 62 scoring matrix with a gap penalty of 12, a gap extend penalty of 4, and a frameshift gap penalty of 5. The percent identity between two amino acid or nucleotide sequences may also be determined using the algorithm of E. Meyers and W. Miller (1989, Cabios, 4: 11-17) which has been incorporated into the ALIGN program (version 2.0) using a PAM120 weight residue table, a gap length penalty of 12, and a gap penalty of 4. Additional search and alignment tools known in the art include, but are not limited to, the NBLAST and XBLAST programs (version 2.0) of Altschul et al (1990) J. Mol. Biol. 215:403-410 and the Gapped BLAST program.

In some embodiments, the polynucleotide cassette of the present invention further comprises an RNA export signal. An RNA export signal is a cis-acting post-transcriptional regulatory element that enhances export of the RNA from the nucleus. Exemplary RNA export sequences include, but are not limited to, sequences from the hepatitis B virus post-transcriptional regulatory element (HPRE) and the woodchuck hepatitis virus post-transcriptional regulatory element (WPRE) (Higashimoto et al (2007) Gene Ther. 14(17):1298-1304), and variants thereof. WPRE is a tripartite element containing gamma, alpha and beta elements in the given order. A shortened version of WPRE containing only minimal gamma and alpha elements may also be used (Choi et al (2014) Molecular Brain 7:17).

In some embodiments, the polynucleotide cassette of the present invention further comprises a polyadenylation region. As is understood in the art, RNA polymerase II transcripts are terminated by cleavage and addition of a polyadenylation region, which may also be referred to as a poly(A) signal, poly(A) region, or poly(A) tail. The poly A region contains multiple consecutive adenosine monophosphates, often with repeats of the motif AAUAAA. Several efficient polyadenylation sites have been identified, including those from SV40, bovine growth hormone, human growth hormone and rabbit beta globin (Xu et al (2001) Gene 272(1-2):149-156; Xu et al (2002) J. Control Reg. 81(1-2):155-163. The most efficient polyA signal for expression of a transgene in mammalian cells may depend on the cell type and species of interest and the particular vector used. In some embodiments of the invention, the polynucleotide cassette comprises a polyA region selected from the group consisting of human growth hormone (HGH), bovine growth hormone (bGH), Simian virus 40 (SV40) and beta-globin. Preferred polyA regions for enhanced expression in mammalian eyes are HGH and bGH.

As used herein, the term “operably linked” refers to a juxtaposition of genetic elements, e.g., promoter, enhancer, termination signal sequence, polyadenylation sequence, Kozak sequence, etc., wherein the elements are in a relationship permitting them to operate in the expected manner. For instance, a promoter is operably linked to a coding region if the promoter helps initiate transcription of the coding sequence. There may be intervening residues between the promoter and the coding sequence or between any two elements so long as the functional relationship is maintained.

The polynucleotide cassette may comprise a scaffold-attachment region (SAR). SARs are known in the art and may include, but are not limited to, a human scaffold-attachment region, and a human SAR from beta-interferon. Scaffold attachment regions (SAR) and matrix attachment regions (MAR) are 70% AT rich sequence, which without being limited by mechanism, have roles in chromatin function. In addition to their structural function SARs and MARs impact temporal and spatial organization of gene expression (Alvarez et al (2000) and Liu et al (1997). Including certain SAR or MAR sequences in a polynucleotide cassette may increase expression of a transgene, but it has been demonstrated that not all SAR and MAR sequences are beneficial for expression of a particular transgene (Sass et al 2005). SARS and/or MARS may reduce silencing via formation of partitioned looped domains and/or by insulating the transgene from positional effects. SARs and MARs may increase gene expression through a mechanism involving increased access to the single stranded DNA by transcription potentiation factors and chromatin remodeling. Further SARs and MARs may stabilize gene expression by anchoring chromatin to the nuclear matrix. Origins of replication complexes tend to form at SAR/MAR regions. The SAR and/or MAR orientation in relation to the transgene impacts expression. SARs and/or MARs may include, but are not limited to, HPRT, Apo8, KSHV and IFN. In some instances, placement of the SAR and/or MAR 3′ to the gene of interest increased expression more than placement 5′ to the gene of interest. In some instances, placement of the SAR and/or MAR 5′ and 3′ to the gene of interest resulted in similar effects on expression. In vitro and in vivo experiments were performed with PRT, Apo8, KSHV and IFN SAR and/or MAR sequences in various orientations. Results obtained from in vitro experiments were not necessarily predictive of results obtained from in vivo experiments (data not shown). For example, the HPRT sequence increased in vitro expression when situated either 5′ or 3′ to the gene of interest. However, when the HPRT sequence was tested in vivo in mice, the HPRT sequence in 3′ to the gene of interest showed a greater increase in expression than when the HPRT sequence was placed 5′ to the gene of interest. Further, expression from cassettes comprising a SAR sequence persisted longer in CHO cells than expression from cassettes lacking a SAR sequence. (Data not shown).

In some embodiments, the gene delivery vector is a recombinant adeno-associated virus (rAAV). In such embodiments, the subject polynucleotide cassette is flanked on the 5′ and 3′ ends by functional AAV inverted terminal repeat (ITR) sequences. By “functional AAV ITR sequences” is meant the ITR sequences function as intended for the rescue, replication, and packaging of the AAV virion. Hence, AAV ITRs for use in the gene delivery vectors of the application need not have a wild-type nucleotide sequence and may be altered by the insertion, deletion, or substitution of nucleotides, or the AAV ITRs may be derived from any of several AAV serotypes including, but not limited to, AAV1, AAV2, AAV3, AAV4, AAV5, AAV6, AAV7, AAV8, AAV9 and AAV10. ITR sequences from AAV may be placed in flanking positions around a polynucleotide cassette of interest for subsequent transfer of the cassette to an AAV genome. A Kozak sequence (for example, GCCACC) may occur 5′ of the start codon of the coding sequence. Preferred AAV vectors have the wild-type Rep and Cap genes deleted in whole or in part but retain functional flanking ITR sequences. In particular embodiments, the AAV viral vector is selected from the group comprising the AAV2 variant 7m8 and the AAV2 variant LSV1.

As will be appreciated by the ordinarily skilled artisan, two or more of the aforementioned polynucleotide elements may be combined to create a polynucleotide cassette of the present disclosure. Thus, for example, the polynucleotide cassette may comprise in operable linkage from 5′ to 3′ order, a CMV enhancer, a CMV promoter, a chimeric intron, a Kozak sequence, a CFI cDNA transgene, a second enhancer region, and a polyadenylation sequence. In some aspects, the chimeric intron may comprise in 5′ to 3′ order TPL, eMLP and IgH. In some aspects, the second enhancer region may comprise a human scaffold attachment region or a WPRE sequence.

In some embodiments, the subject polynucleotide cassette is encapsidated within an AAV capsid, which may be derived from any adeno-associated virus serotype, including without limitation, AAV1, AAV2, AAV3, AAV4, AAV5, AAV6, AAV7, AAV8, AAV9, AAV10 etc, any of which may serve as the gene delivery vector. For example, the AAV capsid may be a wild type or native capsid. Wild type AAV capsids of particular interest include AAV2, AAV5 and AAV9. As with the ITRS, the capsid need not have a wild-type nucleotide sequence but rather may be altered relative to the wild-type sequence by the insertion, deletion or substitution of nucleotides in the VP1, VP2 or VP3 sequence, so long as the capsid is able to transduce mammalian cells. The AAV capsid may be a variant AAV capsid that comprises one or more amino acid substitutions, deletions or insertions relative to the parental capsid protein or AAV capsid protein from which it is derived. Variant AAVs of particular interest may include, but are not limited to, those disclosed in U.S. Pat. No. 9,193,956. In some embodiments, the variant AAV comprises or consists of the 7m8 variant capsid protein (which may be referred to as AAV2.7m8 and 7m8AAV2). In some embodiments, the AAV comprises or consists of an AAV2.5T capsid protein such as provided in U.S. Pat. No. 9,233,131. In some embodiments, the AAV comprises the AAVShH10 or AAV6 capsid protein (U.S. Patent Application Pub. No. 20120164106 and Klimczak et al PLOS One 4(10):e7467 (Oct. 14, 2009)). In some embodiments, the AAV comprises or consists of an AAV2.5T_LSV1 variant disclosed in U.S. Patent App Pub. No. WO2020219933.

In some embodiments, the rAAV is replication defective, in that the AAV vector cannot independently further replicate and package its genome. For example, when cone cells are transduced with rAAV virions, the gene is expressed in cone cells, however, due to the fact that the transduced cone cells lack AAV rep and cap genes and accessory function genes, the rAAV is not able to replicate.

Gene delivery vectors such as AAV virions encapsulating the polynucleotide cassettes of the present application may be produced by any method known in the art and suitable for production of virions for use in mammalian subjects. For example, in the case of AAV virions, an AAV expression vector according to the invention may be introduced into a producer cell, followed by introduction of an AAV helper construct, where the helper construct includes AAV coding regions capable of being expressed in the producer cell and which complement AAV helper functions absent in the AAV vector. This is followed by introduction of helper virus and/or additional vectors into the producer cell, wherein the helper virus and/or additional vectors provide accessory functions capable of supporting efficient rAAV virus production. The producer cells are then cultured to produce rAAV. Replication defective AAV virions encapsulating the recombinant AAV vectors of the instant invention are made by standard techniques known in the art using AAV packaging cells and packaging technology. Examples of such are found in U.S. Pat. Nos. 5,436,146; 5,753,500; 6,040,183; 6,093,570 and 6,548,286. Further methods are provided in US. Patent App. No. 2002/0168342. Helper cells may include, but are not limited to, Sf9 cells and HEK293 cells.

Any concentration of viral particles suitable to effectively transduce mammalian cells can be prepared for contacting mammalian cells in vitro or in vivo. For example, the viral particles may be formulated at a concentration of 10⁸ vector genomes per mL (vg/ml) or more, for example, 5×10⁸ vg/mL; 10⁹ vg/mL, for example, 5×10⁹ vg/ml; 10¹⁰ vg/ml, for example 5×10¹⁰ vg/ml; 10¹¹ vg/ml, for example 5×10¹¹ vg/ml; 10¹² vg/ml, for example 5×10¹² vg/ml; 10¹³ vg/ml, for example 5×10¹³ vg/ml; 10¹³ vg/ml, for example 1.5×10¹³ vg/ml; 10¹⁴ vg/ml, for example 1×10¹⁴ vg/ml and 5×10¹⁴ vg/ml or more, but typically not more than 1×10¹⁵ vg/ml. Similarly, any total number of viral particles suitable to provide appropriate transduction of cells to confer the desired effect or treat the disease can be administered to the mammal.

The subject viral vector may be formulated into a pharmaceutical composition comprising any suitable unit dose of the vector which can be administered to a subject to produce a change in the subject or to treat a disease in the subject. In some embodiments, a unit dose comprises, without limitation, 1×10⁸ vg or more, for example at least about 1×10⁹ vg, 1×10¹⁰ vg, 1×10¹¹ vg, 1×10¹² vg, 1×10¹³ vg, 1×10¹⁴ vg or 1×10¹⁵ vg. In some embodiments a unit dose is from about 1×10⁹ to about 4×10¹² vg/eye, from about 1×10¹⁰ to about 4×10¹¹ vg/eye, from about 2×10¹⁰ to about 3×10¹¹ vg/eye, from about 2×10¹⁰ to about 2×10¹¹ vg/eye, from about 2.5×10¹⁰ to about 2×10¹¹ vg/eye, from about 2×10¹⁰ to about 1×10¹¹ vg/eye, from about 5×10⁹ to about 8×10¹¹ vg/eye, from about 1×10¹¹ to about 2×10¹¹ vg/eye, from about 5×10¹¹ to about 2×10¹¹ vg/eye, or from about 8×10¹⁰ to about 1×10¹¹ vg/eye.

In some cases, the unit dose of a pharmaceutical composition may be measured using multiplicity of infection (MOI). By MOI it is meant the ratio or multiple of vector or viral genomes to the cells to which the nucleic acid may be delivered. In some cases, the MOI may be 1×10⁴ to 1×10⁸, 1×10⁵ to 1×10⁷, or 1×10⁶. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷ and 1×10¹⁸ MOI. In some aspects, the amount of pharmaceutical composition comprises about 1×10⁸ to about 1×10¹⁵ recombinant viruses, about 1×10⁸ to about 10¹⁴ recombinant viruses, about 1×10¹⁰ to about 1×10¹³ recombinant viruses or about 1×10¹⁰ to about 3×10¹² recombinant viruses.

In preparing the subject rAAV compositions, any host cells for producing rAAV virions may be employed including, for example, mammalian cells (e.g., 293 cells), insect cells (e.g., SF9 cells), microorganisms and yeast. Host cells can also be packaging cells in which the AAV rep and cap genes are stably maintained in the host cell or producer cells in which the AAV vector genome is stably maintained and packaged. Exemplary packaging and producer cells are derived from Sf9, HEK293, a549 or HeLa cells. AAV vectors are purified and formulated using standard techniques known in the art.

The present invention includes pharmaceutical compositions comprising a polynucleotide cassette or gene delivery vector described herein and a pharmaceutically-acceptable carrier, diluent or excipient. For example, one embodiment is a pharmaceutical composition comprising a polynucleotide of the present disclosure and a pharmaceutically acceptable excipient. In a specific embodiment, the recombinant virus is a recombinant adeno-associated virus (AAV). The subject polynucleotide cassettes or gene delivery vector can be combined with pharmaceutically acceptable carriers, diluents and reagents useful in preparing a formulation that is generally safe, non-toxic and desirable, and includes excipients that are acceptable for primate use. Such excipients may be solid, liquid, semi-solid or in the case of an aerosol composition, gaseous. Examples of such carriers or diluents may include, but are not limited to, water, saline, Ringer's solutions, dextrose solution and 5% human serum albumin. Supplementary active compounds can also be incorporated into the formulations. Solutions or suspensions used for the formulations may include a sterile diluent such as water for injection, saline solution, fixed oils, polyethylene glycols, glycerin, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation, and compounds for the adjustment of tonicity such as sodium chloride or dextrose. The pH can be adjusted with acids or bases, such as hydrochloride acid or sodium hydroxide. In particular embodiments, the pharmaceutical compositions are sterile.

Pharmaceutical compositions suitable for use with the instant compositions and methods further include sterile aqueous solutions or dispersions and sterile powders for the extemporaneous preparation of sterile injectable solutions or dispersions. In some cases, the composition is fluid to the extent that easy syringe ability exists. In certain embodiments, the compositions are stable under the conditions of manufacture and storage. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant such as pluronic acid 0.001% may be used.

For delayed release, the medicament may be included in a pharmaceutical composition which is formulated for slow release, such as in microcapsules formed from biocompatible polymers or in liposomal carrier systems according to methods known in the art.

In some embodiments the gene delivery vector is administered to the eye of the subject in need of treatment. In some embodiments, the gene delivery vector is administered to the subject via intraocular injection, intravitreal injection, retinal injection, subretinal injection, suprachoroidal injection or by any other convenient mode or route of administration. In some embodiments, the subject is a human subject suffering from or at risk for developing dry-AMD. The eye includes the eyeball and the tissues and fluids which constitute the eyeball and the tissues and fluids which constitute the eyeball, the periocular muscles (such as the oblique and rectus muscles) and the portion of the optic nerve which is within or adjacent to the eyeball. Cell types near retina include, but are not limited to, photoreceptor cells, rod cells, cone cells, retinal pigment epithelium cells and choroid cells.

The term “combination” or the terms “in combination”, “used in combination with” and “combined preparation” as used herein may refer to the combined administration of two or more agents simultaneously, sequentially or separately. The term “simultaneously” as used herein means that the agents are administered concurrently or at the same time. The term “sequential” as used herein means that the agents are administered one after the other. The term “separate” as used herein means that the agents are administered independently of each other but within a time interval that allows the agents to show a combined, preferably synergistic effect.

A tissue “explant” is a piece of tissue that has been transferred from an animal to a nutrient medium.

In some embodiments, the claimed methods result in a therapeutic benefit, e.g., preventing the development of a disorder, halting the progression of a disorder, reversing the progression of a disorder, etc. In some embodiments, the methods comprise the step of detecting that a therapeutic benefit has been achieved. The ordinarily skilled artisan will appreciate that such measures of therapeutic efficacy will be applicable to the particular disease being modified and will recognize the appropriate detection methods to use to measure efficacy.

The methods and compositions of the present application find use in the treatment of dry-AMD. One embodiment is a method of treating dry-AMD in a subject at risk for dry-AMD, the method comprising administering to the subject a gene delivery vector that contains a polynucleotide cassette as disclosed herein, wherein the cassette encodes a human CFI polypeptide effective for reducing one or more signs & symptoms of the medical condition. In some embodiments, the gene delivery vector is an adeno-associated virus, and the polypeptide is a human CFI that is secreted by the cells transduced with by the vector.

In some embodiments, the unit dose of rAAV particles is administered in combination with steroid treatment. In some embodiments, the steroid treatment is a corticosteroid treatment. In some embodiments, the steroid treatment is a systemic steroid treatment. In some embodiments, the steroid treatment is an oral steroid treatment. In some embodiments, the steroid treatment is a prednisone treatment. In some embodiments, the steroid treatment is an ophthalmic steroid treatment. In some embodiments, the ophthalmic steroid treatment is a topical steroid treatment (e.g., a drop), a periocular steroid treatment (e.g., subtenons, subconjunctival), an intravitreal steroid treatment, or a superchoroidal steroid treatment. In some embodiments, the ophthalmic steroid treatment is a glucocorticoid including, but not limited to, an anti-inflammatory glucocorticoid. In some embodiments, the topical steroid treatment is a glucocorticoid including but not limited to, an anti-inflammatory glucocorticoid. In some embodiments, the topical steroid treatment is a difluprednate treatment, a medrysone treatment, a loteprednol treatment, a prednisolone treatment, a fluocinolone treatment, a triamcinolone treatment, a rimexolone treatment, a dexamethasone treatment, a fluorometholone treatment, a fluocinolone treatment, a rimexolone treatment, or a prednisone treatment. Anti-inflammatory glucocorticoids may include, but are not limited to, difluprednate, dexamethasone, prednisolone, triamcinolone, fluorometholone, rimexolone, fluocinolone, loteprednol and bioequivalents thereof. In some embodiments, the topical steroid treatment is a difluprednate treatment. By “dexamethasone” is intended dexamethasone, dexamethasone biosimilars, dexamethasone bioequivalents, and pharmaceutical compositions comprising dexamethasone, a dexamethasone biosimilar or a dexamethasone bioequivalent. Pharmaceutical compositions comprising dexamethasone include, but are not limited to, Ozurdex™, Maxidex™, Decadron™, Dexamethasone Intensol™, Ocu-Dex™, Dexycu™, Dextenza™ and Zodex™. Ozurdex™ is a pharmaceutical composition comprising dexamethasone. By “difluprednate” is intended difluprednate, difluprednate biosimilars, difluprednate bioequivalents, and pharmaceutical compositions comprising difluprednate, a difluprednate biosimilar or a difluprednate bioequivalent. Pharmaceutical compositions comprising difluprednate include, but are not limited to, Durezol™ and difluprednate emulsions. By “triamcinolone” is intended triamcinolone, triamcinolone biosimilars, triamcinolone bioequivalents, and pharmaceutical compositions comprising triamcinolone, a triamcinolone biosimilar or a triamcinolone bioequivalent. Pharmaceutical compositions comprising triamcinolone include, but are not limited to, Triesence™, Xipere™, and Trivaris™. In some embodiments, the steroid treatment is administered before, during, and/or after administration of the unit dose of rAAV particles. In some embodiments, the steroid treatment is administered before administration of the unit dose of rAAV particles. In some embodiments, the steroid treatment is administered during administration of the unit dose of rAAV particles. In some embodiments, the steroid treatment is administered after administration of the unit dose of rAAV particles. In some embodiments, the steroid treatment is administered before and during administration of the unit dose of rAAV particles. In some embodiments, the steroid treatment is administered before and after administration of the unit dose of rAAV particles. In some embodiments, the steroid treatment is administered during, and after administration of the unit dose of rAAV particles. In some embodiments, the steroid treatment is administered before, during, and after administration of the unit dose of rAAV particles.

Methods of determining expression levels are known in the art. Any method of determining expression level may be used in the methods of the application. Methods of determining expression level include, but are not limited to, immunoassay methods and activity assays.

Methods of determining concentration are known in the art. Any method of determining concentration may be used in the methods of the application. Methods of determining concentration include, but are not limited to, immunoassay methods and activity assays.

Immunoassay methods for measuring the presence and quantity of a protein in a biological or cell sample are known in the art. See for example Hage, D. S. (1999) “Immunoassays” Analytica Chemistry 71(12):294-304; The Immunoassay Handbook, 4^{th} Edition: Theory and Applications of Ligand Binding, ELISA and Related Techniques by David Wild (Ed) Elsevier Science (2013). Immunoassays are generally based on the reaction between a target protein and an antibody or antibody fragment specifically binding to the target protein. Immunoassay may be performed in a liquid or solid phase. Suitable immunoassays include, but are not limited to, sandwich and competition assays, Western blotting, ELISAs, radioimmunoassays, fluoroimmunoassays and the like. The biological sample can be a cell culture medium or supernatant (a sample take from the culture without lysing the cells), cell lysate, whole cells, blood, serum, plasma, aqueous humor, vitreous humor or other body fluid or tissue. It is recognized that a biological sample from a subject may be enriched by separation of whole cells from the sample, particularly when the polypeptide of interest may be secreted from a cell. Separation may be by any convenient separation technique known in the art including, but not limited to, fluorescence activated cell sorting (FACS), magnetic separation, affinity chromatography, “panning” with an affinity reagent, centrifugation and ultracentrifugation.

Activity assays are known in the art. Any relevant activity assay may be utilized in the methods of the application. Activity assays may include but are not limited to methods of evaluating C3b-inactivating activity and methods of evaluating iC3b-degradation activity. For example, measurement of CFI proteolytic activity is described in Hsiung et al (1982) Biochem J. 203:293-298. Haemolytic and conglutinating assays for CFI activity are described in Lachmann & Hobart (1978) “Complement Technology” in Handbook of Experimental Immunology 3^{rd} Ed. Ed D M Weir Blackwells Scientific Publications Ch. 5A; and Harrison (1996) “Weir's Handbook of Experimental Immunology 5^{th} ed. Eds; Herzenberg Leonore A., Weir D M, Blackwell C; Blackwell's Scientific Publications Ch.75 36-37. The conglutinating assay is highly sensitive and can be used for detecting both the first (double) clip converting fixed C3b to iC3b and acquiring reactivity with conglutinin; and for detecting the final clip to C3dg by starting with fixed iC3b and looking for the loss of reactivity with conglutinin. The haemolytic assay is used for the conversion of C3b to iC3b, and the proteolytic assay detects all the clips.

By “C3b-inactivating activity” is intended the cleavage of C3b to iC3b or other products. In some aspects, the level of C3b-inactivating activity in the subject, in an eye of the subject, or in the vitreous humor of an eye of the subject is increased to a level that is at least 5%, 10%, 15%, 20%, 25%, 30%, 35%, 40%, 45%, 50%, 55%, 60%, 65%, 70%, 75%, 80%, 85%, 90%, 95% above the level of C3b-inactivating activity prior to administration of the polynucleotide cassette of interest.

By “iC3b-degradation activity” is intended processing of iC3b into a less active or inactive degradation production such as C3dg. In some aspects, the level of iC3b-degradation activity in the subject, in an eye of the subject, or in the vitreous humor of an eye of the subject is increased to a level that is at least 5%, 10%, 15%, 20%, 25%, 30%, 35%, 40%, 45%, 50%, 55%, 60%, 65%, 70%, 75%, 80%, 85%, 90%, 95% above the level of iC3b-degradation activity prior to administration of the polynucleotide cassette of interest.

For example, the level of C3b-inactivating or iC3b-degradation activity in an eye of the subject or in the vitreous humor of an eye of the subject may be increased to a level that is 5-100%, 5-80%, 5-40%, 5-20%, 5-10%, 10-100%, 10-80%, 10-70%, 10-60%, 10-50%, 10-40%, 10-30%, 10-20%, 15-100%, 15-90%, 15-80%, 15-60%, 15-40%, 15-20%, 20-100%, 20-80%, 20-60%, 20-40%, 25-100%, 25-80%, 25-60% or 25-40% above the level prior to administration of the polynucleotide cassette of interest.

In some embodiments, administration of a polynucleotide cassette of interest or vector of interest does not detectably increase the level of C3b-inactivating activity or iC3b-degradation activity in the plasma or serum of the subject. In other embodiments, administration of a polynucleotide cassette of interest or vector of interest does not detectably increase the level of C3b-inactivating activity or iC3b-degradation activity in the plasma or serum of the subject above the level prior to administration of the polynucleotide cassette of interest.

In some instances, the expression of the coding sequence or transgene, as detected by measuring levels of gene product or by measuring therapeutic efficacy may be observed 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 5 months, 6 months, 7 months, 8 months, 9 months, 10 months, 11 months, 12 months, 13 months, 14 months, 15 months, 16 months, 17 months, 18 months, 2 years, 3 years, 4 years, 5 years or more after administration of the subject composition.

The terms “complement factor I cofactor” and “CFI cofactor” refer to a protein that is capable of acting as a cofactor for the CFI-mediated cleavage of C3b.

Methods of visualizing the retina during surgery are known in the art and may be used to identify the retina.

The methods, systems and kits described herein may employ, unless otherwise indicated, conventional techniques and descriptions of molecular biology (including recombinant techniques), cell biology, biochemistry, immunochemistry and virology techniques which are within the skill of those who practice the art. Such conventional techniques include methods for cloning and propagating recombinant virus, formulation of a pharmaceutical composition and biochemical purification and immunochemistry. Specific illustrations of suitable techniques can be had by reference to the examples herein. However, equivalent conventional procedures may also be used. Such conventional techniques and descriptions can be found in standard laboratory manuals such as Weiner et al., Eds, Genetic Variation: a Laboratory Manual (2007); Dieffenbach, Dveksler (Eds.), PCR Primer: a Laboratory Manual (2003); Sambrook and Russell, Condensed Protocols from Molecular Cloning: a Laboratory Manual (2006); Miller & Calos eds. (1987) “Gene Transfer Vectors for Mammalian Cells” and Current Protocols in Immunology (1999-2022) John Wiley & Sons, all of which are hereby incorporated by reference in their entirety for all purposes.

It will be understood that the reference to the below examples is for illustration purposes only and do not limit the scope of the claims. Any of a number of alternative compositions and methods are applicable and suitable for using in practicing the claimed methods and compositions. It is also understood that an evaluation of the expression constructs and methods of the application may be carried out using procedures standard in the art.

### EXAMPLES

### Example 1. Vector Generation

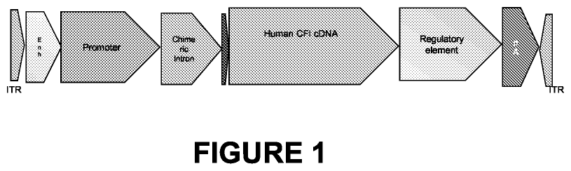

The wild-type human Complement Factor I (CFI) cDNA sequence (1,752 nucleotides—NCBI Reference Sequence: NM_000204.5) as well as five codon-optimized human CFI cDNAs were generated and cloned into the C11 backbone to yield a polynucleotide cassette for enhanced expression of a transgene in the vitreous humor of a mammalian eye. The C11 backbone is described in U.S. patent Ser. No. 11/352,644 and in PCT Application No: PCT/US2018/022996, filed Mar. 16, 2018. A schematic representation of the CFI transgene expression cassette is shown in FIG. 1.

Four of the codon optimized CFI sequences were designed to have improved codon bias for expression and reduced GC content. Nucleotide sequences with lower codon bias scores typically have better expression levels. The codon bias information is summarized in Table 1.

TABLE 1
Codon Bias Score and CpG Numbers of various CFI constructs.

| Name | Codon Bias Score | CpG Number |
|---|---|---|
| CFI_std | 0.27 | 114 |
| CFI_wt | 101.09 | 25 |
| CFI_0.6 | 1.78 | 25 |
| CFI_1.0 | 3.97 | 13 |
| CFI_1.5 | 6.04 | 6 |
| CFI_2.0 | 8.13 | 0(1) |

### Example 2. Plasmid Transfection into OX293 Cells

The day before transfection, Ox293 suspension cells were split at approximately 1.0×10⁶ for a concentration of 1.8×10⁶=2.2×10⁶ cells/ml by the day of transfection. A single well on a 6-well plate containing a total of 3 mL of media was used for each transfection. DNA (3 μg) was diluted in 300 μL of Opti-MEM. Next, 3 μL of TransIT-LT1 Transfection Reagent (Mirus) (1 μL for each mL of media) were added to the DNA/Opti-MEM mixture and incubated for 15 minutes. During incubation, cells were plated in each well at a concentration of 2×10⁶ cells/mL. Transfection mixes were added to wells and incubated on a shaker for 72 hours before supernatant collection. Proteins were secreted into the cell media and CFI protein analysis was performed.

### Example 3. Production of AAV2.5T-LSV1-CFI-1.0 Particles

VPC2.0 cells (ThermoFisher-A49784) were grown in Gibco Viral Production Medium supplemented with 4 mM Glutamax and 8% CO₂. The cells were triple transfected with pAAV ITR-expression (AAV-CFI or AAV CFI-variant), pAAV Rep-Cap and pHelper plasmids and pAAV-ITR expression (AAV-CFI or AAVCFI variant), pAAV Rep, pAAV Cap and pHelper plasmids respectively using AAV-Max transfection. The AAV2.5T-LSV1 serotype variant AAV capsids with improved intravitreal delivery were described in U.S. Patent Publication No: 20200338146, filed Apr. 24, 2020, titled “Variant AAV Capsids for Intravitreal Delivery”. On day 3 post-transfection, cells were lysed in AAV-MAX lysis buffer. The homogenates were cleared from debris by centrifugation. rAAV titer in the digested harvest was measured by ddPCR. The rAAV particles were purified by ultracentrifugation on an iodixanol gradient followed by ultracentrifugation on a CsCl gradient and formulated in the following buffer: 180 mM NaCl, 5 mM NaH₂PO₄, 5 mM Na₂HPO₄, 0.001% poloxamer 188, pH 7.3.

Quality control assays to assess AAV vector identity, purity and functionality were performed. Quality control assays that were performed include SDS-PAGE silver stain, Western-blot anti-VP protein, endotoxin assay, bioburden, alkaline gel and in vitro transduction expression assay; details of some of which are provided below herein. Bioburden analysis of the AAV preparations was tested at Wuxi. Endotoxin analysis of the AAV samples was performed using the Endosafe® nexgen-PTS™ kit.

### Example 4. Production of AAV2-7m8-CFI-1.0 Particles

AAV2.7m8 is a chimeric AAV capsid described in PCT/US2012/034413 filed 20 Apr. 2012. The CFI-1.0 expression cassette was sub-cloned into a Virovek pFastBac (pFB) AAV shuttle vector (see FIG. 1). The resulting clones were sequenced to verify the integrity of the transgene. Recombinant baculovirus were generated. AAV2.7m8-CFI-1.0 was produced in insect Sf9 cells by dual infection with rBV-in2.7m8-RepCap-kozak and rBV-CFI-1.0. The vector was purified with AAVX column purification, followed with iodixanol centrifugation and buffer exchanged to 1×PBS+0.001% pluronic F-68 buffer. The vector was sterilized with 0.22 μm filter. The AAV preparations were tittered by Droplet Digital PCR (ddPCR) as described below herein. Quality control assays to assess AAV vector identity, purity and functionality were performed. Quality control assays that were performed include SDS-PAGE silver stain, Western-blot anti-VP protein, endotoxin assay, bioburden, alkaline gel and in vitro transduction expression assay; details of some of which are provided below herein. Bioburden analysis of the AAV preparations was tested at Wuxi. Endotoxin analysis of the AAV samples was performed using the Endosafe® nexgen-PTS™ kit.

### Example 5. Droplet Digital PCR (ddPCR)

Digested harvested rAAV were diluted 1/5000 folds by dilution buffer with 0.05% Pluronic F-68 (Gibco, Invitrogen, Grand Island, NY). The reaction mixtures were assembled using a ddPCR Supermix for Residual DNA detection kit (BIO-RAD, Hercules, CA) with 0.9 μM primers and 0.1 μM probe, in a final volume of 25 μL as stated in the manufacturer's protocol. ddPCR was done as described in ddPCR amplification guide (Bio-Rad 6407). Test samples were emulsified with droplet generator oil using a QX-100 droplet generator (BIO-RAD) according to the manufacturer's instructions. PCR amplification of the droplets was performed using a conventional thermal cycler with the following parameters: 95° C. for 10 minutes, followed by 39 cycles of 94° C. for 30 s and 60° C. for 30 s, followed by a final 98° C. heat treatment for 10 minutes. The PCR plate was subsequently scanned using a QX200 droplet reader (BIO-RAD) and the data were analyzed with QuantaSoft software (BIO-RAD). The primers and probe used for the analysis are described in Table 2.

TABLE 2
ddPCR Primers and Probe

| | | |
|---|---|---|
| CMV-F | TGA GTC AAA CCG CTA TCC | SEQ ID NO: |
| | AC | |
| CMV-R | TGC CCA GTA CAT GAC CTT | SEQ ID NO: |
| | AC | |
| CMV-FAM | ACT GCC AAA ACC GCA TCA | SEQ ID NO: |
| | GC | |

### Example 6. Alkaline Gel Analysis of AAV-CFI-1.0 Packaged into AAV2.7m8 and AAV2.5T-LSV1

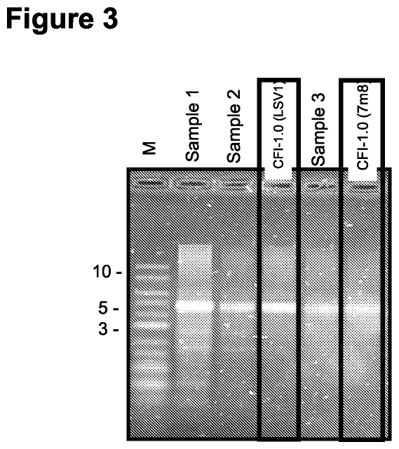

Samples of each AAV preparation (2×10¹⁰ vg max) were mixed with 6× Alkaline loading buffer, incubated 5 minutes at 95° C., cooled down on ice and loaded on a 1% agarose alkaline gel. The gel was set to run at 30 Volts for 14 hours in 1× Alkaline buffer. The gel was washed with 1×TBE for 45 minutes then stained with 1×SyBr gold for 60 minutes. The gel was washed with water and imaged with a gel imager. A representative image is shown in FIG. 3.

### Example 7. AAV Transduction of HEK293T Cells

HEK293T cells were seeded in DMEM+GlutaMAX supplemented with 10% HI-FBS, 1×NEAA and 1 mM MgCl2 at 2×10′ cells per well in a 24-well plate. The media was replaced with BalanCD supplemented with 2% GlutaMax before performing the transduction. The cells were transduced in duplicates at 2E4 MOI (multiplicities of infection) and incubated for ~72 hours. The supernatant was harvested in 1.5 mL tubes approximately 72 hours post transduction and centrifuged at 4° C. for 5 minutes at 500×g. The supernatant was transferred in new 1.5 mL tubes and stored at −80° C.

### Example 8. AAV Transduction of Rabbit Explants

Rabbit explants were obtained from Pacific Biolabs. Retinas were carefully dissected from the adult rabbit eyes 2-3 hours post-euthanasia. A 6 mm biopsy punch of retina was flatted (photoreceptor side downward and ganglion cell side upward) in the transwell membrane (Corning transwell polycarbonate membrane cell culture inserts, cat #: CLS3401-48EA). The explants were incubated with complete media in 12-well plates. The explants were transduced with the AAV-CFI-1.0 vector packaged into AAV2.7m8 at 2.5E10 MOI and at 5E10 with the AAV-CFI-1.0 vector packaged into AAV2.5T-LSV1 in transduction media. The vector cocktail was transferred from beneath the transwell to the top of the explants every 30 minutes for 2 hours. Complete neurobasal media (400 μl) supplemented with GlutamaxÔ, B27, and Antibiotic-Antimycotic (Anti-Anti) was added to the bottom of the wells of the 12-well plate. The supernatant was collected every 2-3 days, stored at −80° C. and replaced with neurobasal media supplemented with GlutamaxÔ, B27, and Anti-Anti for 14 days. The explants were collected, rinsed twice with PBS and stored at −80° C.

### Example 9. CFI Western Blot Analysis

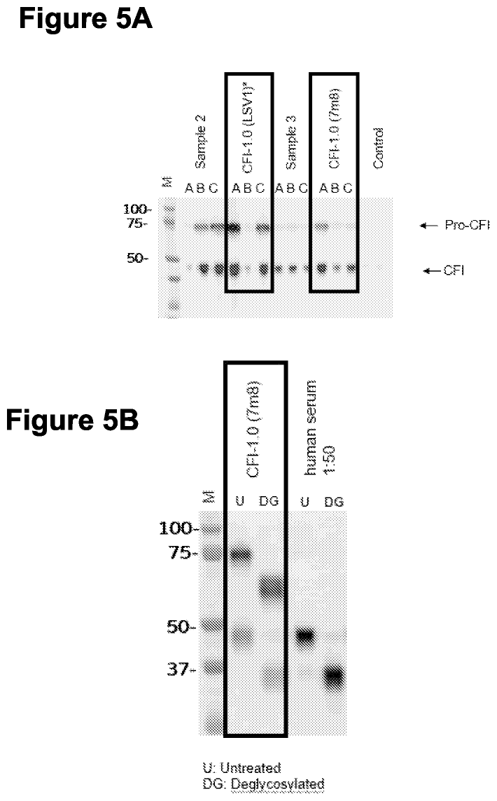

Harvested supernatant (20 μl) was mixed with 5× reducing buffer (5 μl) and incubated at 95° C. for 10 minutes 20 μl was loaded on a 4-12% Bis-Tris gel with 1×MOPS running buffer followed by semi-dry blotting. The membrane was incubated in the blocking buffer (1% milk in 1×TBST) for 1 hour on a rocker at room temperature followed by an overnight incubation with a monoclonal human CFI antibody on a rocker at 4° C. An anti-CFI antibody from CusaBio was used for the Western blot performed for the transfected cells (1:2000 dilution, CSB-PA005279LA01HU) (FIG. 2B). For the Western blot performed for the AAV transduced cells, an anti-CFI antibody from Abcam was used (1:2000 dilution, ab278524). The membrane was washed 4×5 minutes with 1×TBST and incubated 1 hour at room temperature with goat anti-rabbit IgG HRP antibody (Cell Signaling, 7074S). The membrane was washed 4×5 minutes with 1×TBST, incubated in the developing reagent for 1 minute and visualized with the Amersham ImageQuant 800 imaging system. Representative images are shown in FIG. 5A and FIG. 5B (both 5 sec exposures).

### Example 10. CFI Functional Assay

The C3b alpha chain is degraded by CFI into iC3b. iC3b can be detected by immunoblotting as a reduction of the alpha chain at 116 kDa and appearance of the two iC3b breakdown bands at 68 kDa and 43 kDa.

Rabbit eye explants were transduced in duplicate using the following amounts of virus (for AAV2.7m8-CFI-1.0-2.5E10 vg/explant, for AAV2.5T-LSV1-CFI-1.0-5E10 vg/explant). Cell media was collected at day 11 after rabbit eye explant transduction with AAV2.7m8-CFI-1.0 and AAV2.5T-LSV1-CFI-1.0 vectors. Equal volumes were loaded in each functional assay experiment (1/50 dilution of supernatant).

The cell culture media supernatant was diluted 1:50 in PBS. 16 μl of the 1:50 dilution was mixed with 0.5 μg of purified CFH (Complement Technology, Inc. #A137) and 1.0 μg of purified C3b (Complement Technology, Inc. #A114). A positive control contained 1.0 μg purified C3b, 1.0 μg purified CFI, and 0.5 μg purified CFH. A negative control contained 1 μg purified C3b and 0.5 μg purified CFH. All samples and controls were incubated for 1 hour at 37° C. 5× reducing buffer was added, followed by a 10 minute incubation at 95° C.

20 μl of the reaction was loaded on a 4-12% Bis-Tris gel with 1×MOPS running buffer followed by semi-dry blotting. The membrane was incubated in the blocking buffer (1% milk in 1×TBST) for 1 hour on a rocker at room temperature followed by an overnight incubation with a human C3 antibody (1:2000 dilution, AbD Serotec/BioRad, #AHP1752) on a rocker at 4° C. The membrane was washed 4×5 minutes with 1×TBST and incubated 1 hour at room temperature with an anti-goat IgG HRP antibody (Jackson Immuno, #705-035-003). The membrane was washed 4×5 minutes with 1×TBST, incubated in the developing reagent for 1 minute and visualized with the Amersham ImageQuantÔ 800 imaging system.

### Example 11. Non-Human Primate (NHP) Evaluation of AAV-Mediated Expression of Human Complement Factor I (hCFI) (AAV-CFI 1.0)

AAV-CFI1.0 was evaluated in a nonclinical, non-GLP study in non-human primates (NHPs) designed to evaluate the safety, efficacy, and pharmacokinetics of human CFI expression following an intravitreal (IVT) administration of AAV-CFI-1.0. This species was selected for these studies because in NHPs, the overall retinal structure, including the presence of a fovea, closely resembles that of humans (Picaud Proc Natl Acad Sci USA. 2019 Dec. 26; 116(52): 26280-26287). The study consisted of three groups of male cynomolgus monkeys approximately 2.5 years old and weighed 1.9-2.1 kgs at study start. The animals (n=3 per group) received a single bilateral IVT administration of AAV-CFI-1.0 at 3E10 vg/eye or 1E11 vg/eye or vehicle with a fixed dose volume of 50 μL/eye. The animals were observed until study termination (Day 88). No anti-inflammatory steroids were used in the study.

The following parameters and end points were evaluated in this study: mortality, clinical observations, qualitative food consumption, body weight, ophthalmic examinations, intraocular pressure, electroretinography, and optical coherence tomography. Whole blood, serum, and tissues were collected during the study for possible exploratory analyses. Vitreous humor and aqueous humor were collected at Day 28, 62 and 88.

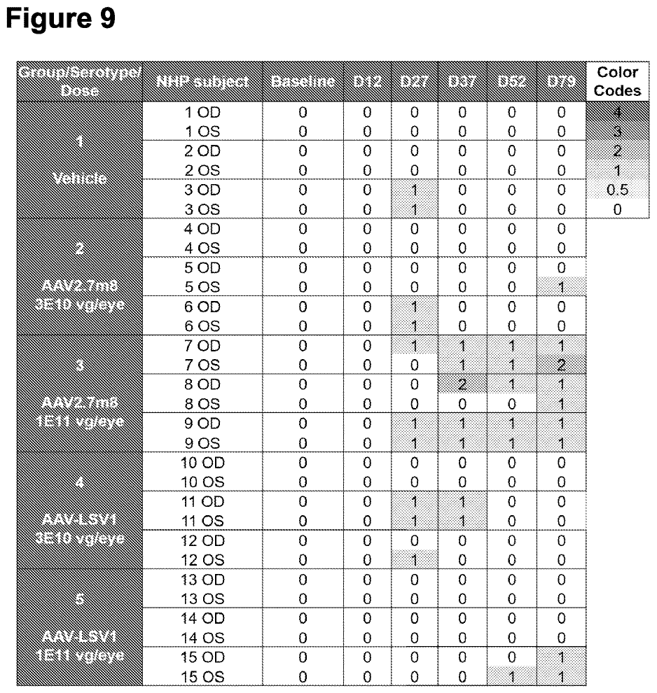

Intravitreal administrations of AAV-CFI-1.0 packaged into both AAV2.7m8 and AAV2.5T-LSV1 capsid serotypes were well tolerated. Test article-related observations were limited to mild self-resolving intraocular ocular inflammation in some of the dosed eyes. The observations included self-resolving minimal to moderate anterior chamber cells in 2 of 3 animals (4 out of 6 eyes total) in 1E11 vg/eye dose group 3, as well as transient minimal to moderate vitreous cells seen in 1 of 3 animals (1 of 6 eyes total) in group 2 (3E10 vg/eye), 3 of 3 animals (6 eyes of 6 total) in group 3 (1E11 vg/eye); 2 of 3 animals (3 eyes of 6 total) in group 4 (3E11 vg/eye), and 1 of 3 animals (2 eyes of 6 total) in group 5. Vitreous cell infiltrates were also transiently seen in vehicle treated animal group (Group1), in 1 of 3 animals (2 eyes of 6 total) indicating the procedure-related effect. Vitreous cell clinical scores are shown in FIG. 9.

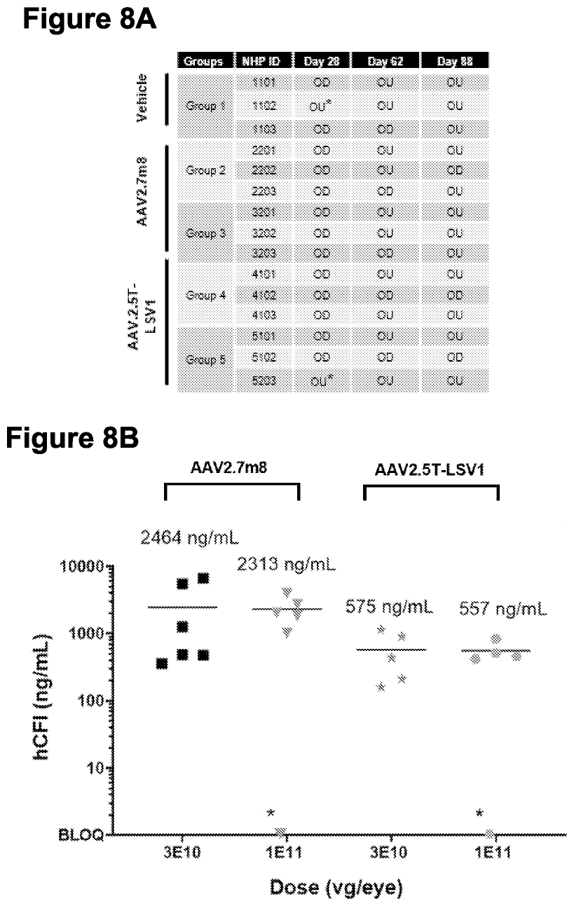

Administration of AAV-CFI-1.0 at both doses resulted in meaningful human CFI levels. Results from one such series of experiments are shown in FIG. 8B.

In conclusion, administration of AAV-CFI-1.0 by a single bilateral intravitreal injection was well tolerated in cynomolgus monkeys at levels of 3E10 vg/eye or 1E11 vg/eye (Human equivalent doses of 6E10 vg/eye or 2E11 vg/eye taking into consideration vitreous volume 2× difference between NHP and human). Slight or mild self-resolving ocular inflammation characterized by vitreous and anterior chamber cell infiltrates were considered AAV-CFI-1.0 related and non-adverse. At 6E10 vg/eye, approximately 2500 ng/ml of human CFI in the vitreous humor is expected using AAV2.7m8 capsid.

### Example 12. CFI Luminex Assay for hCFI Analysis from Vitreous Humor (VH)

NHP vitreous humor samples were collected and snap-frozen on dry ice. For analysis, vitreous samples were thawed on wet ice, vortexed and spun down in preparation for assaying. MILLIPLEX Human Complement Panel 1 Kit (#HCMP1MAG-19K, Millipore) was used to analyze the expression of CFI in vitreous humor. On Day 1 of assay, samples were diluted 25-fold using 4 μL of vitreous humor sample in 96 μL of assay buffer provided by the kit. Standard controls were prepared by serially diluting the kit calibrator in 4% vitreous humor in assay buffer. The standard curve used for interpolating results incorporates concentrations of 167-0.69 ng/mL, with a 3-fold serial dilution. The vitreous humor used in standard control preparation is commercially procured (Pooled Cynomolgus Vitreous Humor, BioIVT, #NHP01VITHUM). Magnetic Luminex beads, conjugated to CFI, were prepared by combining 150 μL of bead stock to 2.85 mL of bead diluent. 200 μL of 1× wash buffer (provided by the kit) was pipetted into each well and plate was set to shake at room temperature at 600 RPM for 10 minutes. Contents were then decanted from within the plate and tapped dry on an absorbent towel. The bead mixture was vortexed and plated 25 μL in each well, along with 25 μL of standard control, 25 μL of diluted sample, and 25 μL of assay buffer. The plate was set to incubate at 4° C., shaking at 600 RPM for 16-18 hours. On Day 2 of the assay, plate was washed with 1× wash buffer for 3 cycles and using a handheld magnet for washing. 50 μL of primary detection antibodies were added into each well and incubated at room temperature, shaking at 600 RPM for 1 hour. After that incubation elapsed, 50 μL of Streptavidin-Phycoerythrin in each well, without washing or decanting previous contents. This incubation took place at room temperature on a shaking plate at 600 RPM for 30 minutes. This was followed by then washing the plate for 3 wash cycles using the handheld magnet and adding 150 μL of Drive Fluid Plus (#4050030, Luminex) into each well of the plate. The plate shook for 5 minutes at 600R RPM, at room temperature. The plate was then read using FLEXMAP 3D^{Ò} Luminex instrument. The readout used for analysis was Median MFI raw data which was then imported into SoftMax Pro 7.1.2 for final analysis. The % CV (Coefficient of Variation) for the standard controls shall be less than or equal to 20% and the recovery should be within ±20%. If more than 2 out of 7 points fail this criterion, the assay was deemed to be invalid.

### Example 13. CFI Luminex Assay for hCFI Analysis from Ocular Tissue Lysates

### Tissue Lysis

Prepare Lysis Buffer necessary for hCFI protein extraction from ocular tissues (10 mL of HEPES Buffer containing 1% Triton X-100, 1 tablet of Mini Protease Inhibitor (EDTA free), and 100 μL of 100×PMSF solution). Vortex contents to create a homogeneous mix, scale volumes accordingly. Using Precellys Lysing Kit Bead for Soft Tissue (Bertin Corp, P000933-LYSK0-A, 2 mL size), place the tubes containing beads on wet ice or chill them at 4° C. Place Lysis Buffer on ice to maintain the temperature to be at 4° C. Add 25× volume to weight of chilled Lysis Buffer onto ocular tissues inside tubes. The ocular tissues analyzed are retinal, Iris/ciliary body, and choroid tissues. i.e., if weight of tissue is 20 mg, 500 μL of Lysis Buffer is added onto tissue. This is 25× more volume than initial tissue weight. Once added, incubate tissues in Lysis Buffer for a minimum of 30 minutes on wet ice. Vortexing samples throughout incubation periodically. Using the Precellys Homogenization with Cryolys Evolution instrument, place the tubes containing Lysis Buffer and tissue inside the machine and use the following settings: Spin: 5000 RPM, Cycles: 4×30 seconds, Break: 15 seconds. Load the Precellys instrument with dry ice. Press start and wait for all samples to be properly homogenized by the machine. After cycles are finished, remove tubes and centrifuge samples for 15 minutes at 2-8° C. at 14,000×g. If debris is seen within supernatant, transfer all contents to a clean low-bind protein tube and perform a quick-spin for 1 minute at 14,000×g to pellet debris. Transfer supernatant to clean low binding protein tube. Store samples at 2-8° C. while testing or freeze down samples at −80° C. for long term storage. Save pellet in bead tubes, store them at −80° C. for long term storage.

hCFI Luminex Assay for Tissue Lysates Analysis

Tissue lysates were analyzed via MILLIPLEX Human Complement Panel 1 Luminex Kit (#HCMP1MAG-19K, Millipore) for levels of human CFI expression. Ocular tissue lysates were vortexed and spun down in preparation for assaying and kept on wet ice. On Day 1 of assay, retinal lysate samples were tested neat using 60 μL of sample, Iris/ciliary body lysate samples were tested using a 2-fold dilution, using 30 μL of lysate sample and 30 μL of assay buffer (provided by the kit), choroid lysate samples were tested using a 10-fold dilution, incorporating 6 μL of lysate sample and 54 μL of assay buffer.

Standard controls were prepared by serially diluting the kit calibrator in 100% assay buffer first, then loading 100% naïve cynomolgus retina lysate prepared using a 25× weight to volume ratio for retina lysate matrix, 50% iris/ciliary body naïve cyno lysate prepared using a 25× weight to volume ratio, and 10% choroid from naïve cynomolgus NHP tissue for choroid lysate matrix. All naïve cynomolgus ocular tissues were sourced from BioChemed Pharmacological, Inc. The standard curve used for interpolating results incorporates concentrations of 500-0.69 ng/mL, with a 3-fold serial dilution. Magnetic Luminex beads, conjugated to CFI, were prepared by combining 150 μL of bead stock to 2.85 mL of bead diluent. 200 μL of 1× wash buffer (provided by the kit) was pipetted into each well and plate was set to shake at room temperature at 600 RPM for 10 minutes. Contents were then decanted from within the plate and tapped dry on an absorbent towel. The bead mixture was vortexed and plated 25 μL in each well, along with 25 μL of standard control, 25 μL of diluted sample, and 25 μL of assay buffer and/or appropriate lysate matrix.

The plate was set to incubate at 4° C., shaking at 600 RPM for 16-18 hours. On Day 2 of the assay, plate was washed with 1× wash buffer for 3 cycles and using a handheld magnet for washing. 50 μL of detection antibodies were added into each well and incubated at room temperature, shaking at 600 RPM for 1 hour. 50 μL of Streptavidin-Phycoerythrin was added in each well, without washing or decanting previous contents. The plate was incubated at room temperature on a shaking plate at 600 RPM for 30 minutes. This was followed by washing the plate for 3 wash cycles using the handheld magnet and adding 150 μL of Drive Fluid Plus (#4050030, Luminex) into each well of the plate. The plate shook for 5 minutes at 600R RPM, at room temperature. The plate was then read using FLEXMAP 3D Luminex instrument. The readout used for analysis was Median MFI raw data which was then imported into SoftMax Pro 7.1.2 for final analysis. The % CV (Coefficient of Variation) for the standard controls shall be less than or equal to 20% and the recovery should be within ±20%. If more than 2 out of 7 points fail this criterion, the assay was deemed to be invalid.

### Example 14. AAV Vector Genome (Vg) DNA Analysis

Serum and tissue samples were collected from NHP after intravitreal (IVT) injection of AAV-CFI-1.0 vector packaged into either the AAV2.7m8 or AAV2.5T-LSV1 capsid serotypes.

NHP tissues collected at termination of the in life part of the study intended for vector genome DNA analysis were snap frozen on dry ice and stored at −80° C. Tissues for the vector DNA biodistribution analysis included: ciliary body with iris (right and left), cornea (right and left), choroid (right and left), lens (right), optic nerve (right and left), lateral geniculate nucleus (left), optic chiasm (left part), optic tract (left), spleen, salivary glands (right and left), lymph nodes (left mandibular and mesenteric), eyelid (right and left), liver (left lateral lobe), heart (left ventricular), kidney (right and left), lung (left caudal lobe), testis (right and left), cerebellum (left), and visual cortex (right and left). For vector genome DNA analysis, whole blood was collected to venipuncture to K2EDTA tubes and frozen over dry ice or in a freezer set to maintain −80° C., within 60 minutes of blood collection. Blood samples were placed on dry ice within 60 minutes of collection and stored in a freezer set to maintain 80° C. until analysis.

### Whole Blood and Tissue Preparation

This entire workstream was performed by Avance Bioscience. DNA extractions were performed for 467 tissue samples. The concentration of the extracted DNA samples was measured using a Cytation 5 Imaging Reader via PicoGreen. The extracted DNAs were stored at −20° C. before qPCR analysis.

### Standard Curve Samples

Standard Curve dilution series was included in each Adverum CMV qPCR assay reaction plate. Eight standard curve dilutions of Control DNA were prepared using the linearized control plasmid DNA at the concentrations ranging from 25 to 1E7 copies/reaction. All the standard curve dilutions consisted of 500 ng of NHP liver genomic DNA and were run in duplicate per reaction.

### No Template Controls (NTC)

No Template Controls (NTC) were included in each qPCR plate in triplicate wells as a negative control to assess potential contamination of the reaction components. The diluent, PolyA/TE buffer, was used as the NTC.

### Negative Control Samples

Negative controls consisting of 500 ng of NHP liver genomic DNA (NEG) were prepared and were run in triplicate wells.

### Spike Control

DNA generated from the linearized control plasmid was used to test for PCR inhibition. 2E5 copies per reaction of the linearized DNA were spiked into the third-well of each sample to assess PCR inhibition. Duplicate spike control wells with 2E5 copies per reaction of the linearized DNA and 500 ng naive NHP liver genomic DNA were also included in each plate.

### Assay Control

The assay control (AC) sample consisting of 500 ng of naïve NHP liver gDNA and 2E+5 copies of control plasmid DNA was included on each qPCR plate in triplicate wells to test the assay performance. The prepared AC samples were aliquoted and stored at −20° C.

qPCR Assay Plate Setup and Amplification

Reactions were assembled in PCR workstations in dedicated PCR laboratories. Thermal cycling and data collection were performed on the Applied Biosystems QuantStudio 7.

### Pro Real-Time PCR System.

After each qPCR run, the data were analyzed using QuantStudio™ Design and Analysis Software v 2.5.0 and the Cₜ values of each reaction were determined with a Cₜ threshold set in the exponential phase of amplification. The copies of target DNA (Mean Quantity) were calculated by averaging the quantities detected in the first two wells for each sample. The Cₜ value of the third well containing a spike for each sample was examined to determine whether the Cₜ value of each third well was one Cₜ (≥1 Cₜ) higher than the spike control to assess PCR inhibition. The number of target copies per reaction, if equal to or above the LOQ (25 copies/reaction), was calculated and reported. Samples with measured quantity below the LOQ are reported as <LOQ.

Each reaction plate was evaluated according to the following acceptance criteria:
Target amplification is detected in the standard curve by qPCR for the target assay with a coefficient of determination (R²)≥0.99 and a slope of −3.1 to −3.6.
For each standard, the difference between the duplicate Cₜ values must be less than 1.0. For the 8-point standard curve, up to two outlier points may be excluded from calculation to improve the standard curve slope and fit for more accurate quantitation, with the exception of Std1 and Std8.
The average Cₜ for the NTC must be greater than the average Cₜ of the LOD (10 copies per reaction) as determined by the assay development.
The Cₜ values of replicate wells for each test sample must be within 1 Cₜ of each other, if within the qualified standard curve range of quantitation (25 copies/reaction to 1E7 copies/reaction), to be qualified for test sample quantitation calculation.
The Cₜ values of the third well, test sample with DNA spike, must be no more than one Cₜ value greater than the spike control Cₜ value to be considered to have uninhibited PCR.
qPCR data were evaluated according to the acceptance criteria above. qPCR plates used for the project data analysis had a qualified standard curve dilution point Std8 (the LOQ) with replicate reactions differing by less than 1 Ct value, thereby meeting the acceptance criteria. All the qualified plates had their standard curve slopes within −3.1 to −3.6 and coefficients of determination (R²) greater than 0.99, meeting the acceptance criteria.
   All sample test results reported in this report met the criteria listed above. The sample test results were valid.

### Example 15. Ocular Compartment Analysis

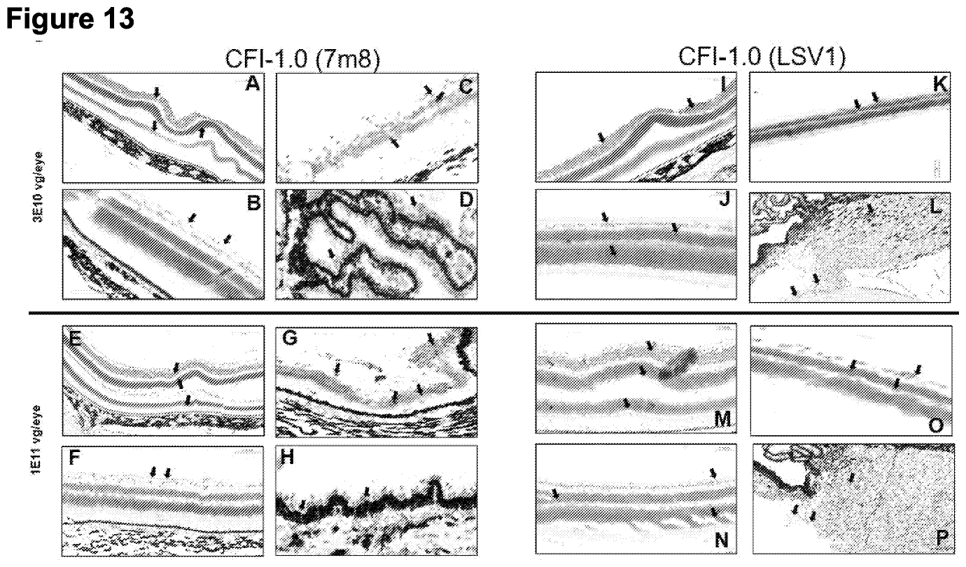

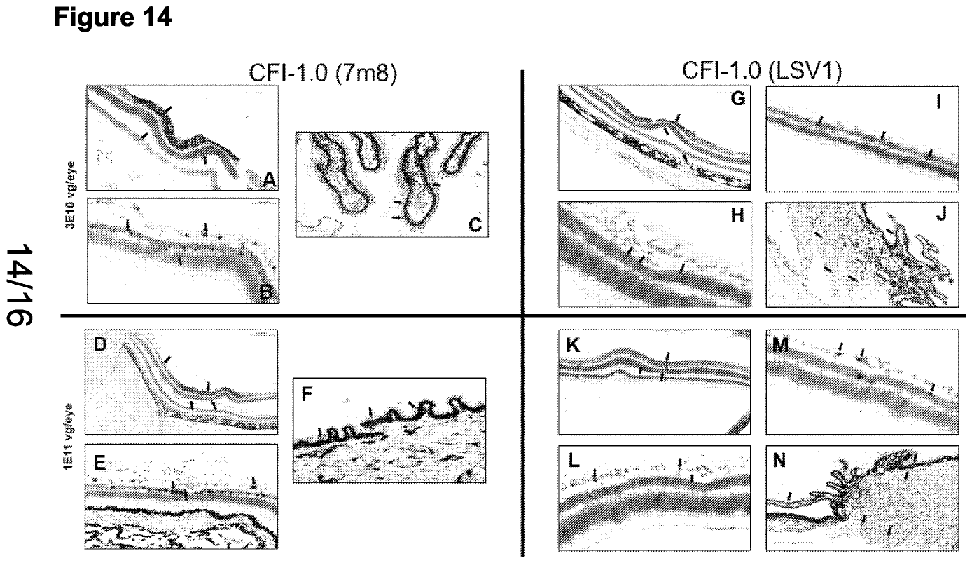

The ocular compartment of NHPs were analyzed after intravitreal (IVT) injection of AAV-CFI-1.0 vector packaged into either the AAV2.7m8 or AAV2.5T-LSV1 capsid serotypes. This workstream was mostly performed by Advanced Cell Diagnostics (ACD) bio. In general, AAV-CFI vector DNA and mRNA microscopic distribution in ocular compartments were evaluated using BaseScope In-Situ Hybridization Methods (ISH). For the analysis, upon eye enucleation, the cornea, iris and lens were removed from the globes. The remaining posterior segment and isolated anterior structures were fixed in 10% neutral buffered formalin for 24 hours and stored in 70% ethanol up to 72-hours. The tissues were prepared for paraffin embedding using vacuum infiltration processing and placed into paraffin blocks and stored at 4° C. until sectioning. Transverse sections of the posterior segment were collected through the macula at 5-μm thickness and mounted on charged slides. Transverse sections of the isolated anterior structures were collected at 5-μm thickness and mounted on charged slides. Slides were stored at 4° C. until shipment to ACD bio. Ten sequential slides were shipped to ACD for subsequent ISH analysis. ISH was performed using the BaseScope LS Red Reagent Kit (Cat. No. 323600) following optimized pretreatment conditions which included epitope retrieval of 15 minutes at 88° C., and protease III treatment for 15 minutes at 40° C. ISH labeling of AAV-CFI vector genome DNA was performed using a 1-ZZ paired probe to the sense of the CMV promoter. Results from one such experiment are shown in FIG. 13. Labeling of AAV-CFI derived mRNA was performed using a 1-ZZ paired probe complementary to the splicing junction, generated after the intron has been spliced from mRNA during maturation, which allows for the detection of AAV-CFI expressed mRNA without DNA interference. Results from one such experiment are shown in FIG. 14. Counterstain of Gill's hematoxylin for 2 minutes at room temperature was performed after probe, amplification, and chromogen steps in the LS kit assay.

### Example 16. Anti-Drug Antibody (ADA) Formation Assay

This workstream was performed by Charles River Laboratories. In general, all reagents and samples were removed from storage and equilibrated to room temperature. 30 μL of Positive/Negative Control/Sample were added to cluster tubes or dilution plate per duplicate set. The High Positive Control (HPC) had a concentration of 2500 ng/mL of hCFI recombinant protein, the Low Positive Control (LPC) had a concentration of 5000 ng/mL, the Negative Control (NC) was Pooled Cyno Serum. 120 μL/well of Acid Diluent (100 mM Glycine-HCl, pH 2.5) was added to dilution plate or cluster tubes. Covered with a plate sealer and incubated shaking at 600 RPM, room temperature, for 30 minutes. Conjugate diluent (0.2 M Tris-HCl in Assay Buffer) was prepared by diluting Neutralization Buffer (1 M Tris HCl, pH 9.5) 5-fold in Assay Buffer (1% BSA in 1×PBS). Master mix was prepared by diluting biotin labeled hCFI recombinant protein (Sigma-Aldrich, #C5938-.1MG) and Sulfo-Tagged hCFI recombinant in conjugate diluent to 1 μg/mL respectively. The Biotin-hCFI and Sulfo-Tagged-hCFI both underwent a 400-fold dilution within the master mix. Next, 100 μL/well of master mix was added to new dilution plate. 50 μL/well of diluted samples was transferred to the new dilution plate containing the master mix. One well for each duplicate was set, which totals to a 15-fold final Minimum Required Dilution (MRD). Dilution plate was covered with a plate sealer and incubated shaking for 2 hours at room temperature at 600 RPM. At the end of incubation, 200 μL/well of Assay Buffer was added to a separate MSD streptavidin plate (Meso Scale Discovery, #L15SA-1). This plate was covered with a sealer and incubated shaking for 1-2 hours at 600 RPM, room temperature. This MSD plate was washed 3 times with 300 μL/well of Wash Solution (0.05% Tween 20 in 1×PBS), using an automated plate washer. Taped dry. 50 μL/well was transferred from dilution plate to the MSD streptavidin plate in duplicate. A plate sealer was used to cover and incubate shaking for 1 hour at 600 RPM, room temperature. Equal amounts of 4× Read Buffer and HPLC Grade Water were added. MSD streptavidin plate was washed 3 times with 300 μL/well of Wash Solution, using an automated plate washer. Taped dry. 2× Read Buffer (150 μL/well) was added to plate and read on MSD QuickPlex SQ 120 instrument.

### Acceptance Criteria

The control acceptance criteria for LPC and HPC are as follows, the % CV (Coefficient of Variation) for both shall be less than or equal to 20%. The ECL signal should be less than or equal to PSCP (Plate-Specific Cut Point) for the LPC, but less than or equal to electrochemiluminescence (ECL) of LPC for the HPC requirement. Each bioanalytical run was accepted if at least 3 out of 4 PC's meet the acceptance criteria for precision (% CV) and ECL. The control acceptance for negative controls were as such, at least 2 of the 3 duplicate sets of NC must have a precision between duplicate wells of less than or equal to 20%. The sample acceptance criteria was as follows, the LPC and HPC will be analyzed 2 times in duplicate on each screening plate. Study samples were assayed once in duplicate wells. The NC was assayed 3 times in duplicate on each plate. The median ECL of the 6 wells (3 duplicate sets) of NC was used to calculate the PSCP. A normal screening multiplication factor was determined during qualification testing and was multiplied by each plate median NC ECL signal to calculate each individual PSCP. Each sample analyzed was compared to the PSCP.

The cut point was statistically determined to be a multiplication factor of 1.332. The PSCP is equivalent to this CP factor multiplied by the median NC signal on the plate tested. The data analyzed in this ADA assay implements a 0.1% false positive rate, incorporating a 6× Standard Deviation calculation. For the first plate of samples, the PSCP resulted to be 85 ECL while the second plate had a PSCP of 69 ECL. Any ECL signal higher than these PSCP values were deemed to be positive for ADA.

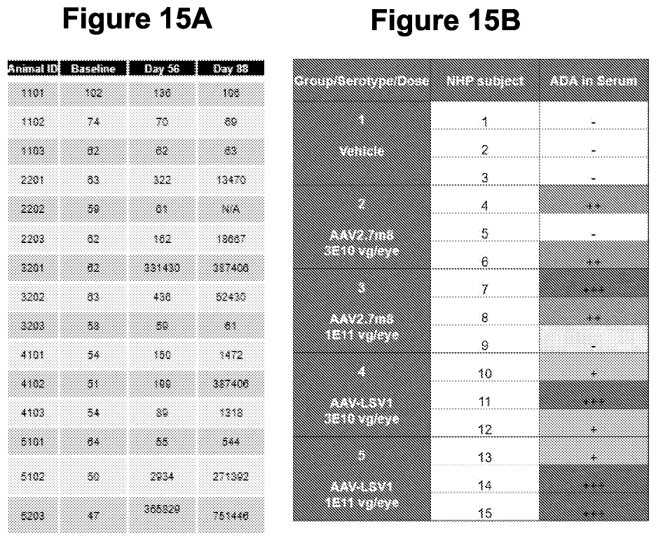

The results are shown in FIGS. 15A and 15B and demonstrate the presence of anti-drug antibodies in serum of most NHPs.

### Example 17. Non-Denaturing Protein Deglycosylation Assay

Supernatant collected from transduced HEK293T in balanCD media (BalanCD HEK293: VWR Cat No: MSPP-91165-1) containing 2% glutaMax was diluted 1:2. 16 ul of the 1:2 dilution was mixed with 2 ul of 10× deglycosylation mix buffer 1 and 2 ul of protein deglycosylation mix II (NEB; P6044), incubated at 25° C. for 30 minutes, then incubated at 37° C. for 16 hours. Naïve human serum was diluted 1:50. 16 ul of 1:50 dilution was mixed with 2 ul of 10× deglycosylation mix buffer 1 and 2 ul of protein deglycosylation mix II, incubated at 25° C. for 30 minutes, then incubated at 37° C. for 16 hours. 5× reducing buffer was added, followed by a 10-minute incubation at 95° C.

Untreated samples: The supernatant from transduced cells (16 ul of 1:2 dilution) and the human serum (16 ul of 1:50 dilution) were mixed with 4 ul of PBS instead of the deglycosylation mix buffer 1 and protein deglycosylation mix II. 5 ul of 5× reducing buffer was added, followed by a 10-minute incubation at 95° C. The reactions were loaded on a 4-12% Bis-Tris gel with 1×MOPS running buffer followed by semi-dry blotting. The Western blot assay was performed as described in Example 9. Representative image is shown in FIG. 5B.

### Example 18. AAV Vector mRNA Analysis

This workstream was performed by Avance Bioscience. Generally, tissue samples were collected from NHP after intravitreal (IVT) injection of AAV-CFI-1.0 vector packaged into either the AAV2.7m8 or AAV2.5T-LSV1 capsid serotypes.

NHP tissues collected at termination of the in life part of the study were snap frozen on dry ice and stored at −80° C. Tissues for mRNA biodistribution analysis included: right retina; right choroid/retinal pigment epithelium (RPE); left cornea; right iris/ciliary body; right optic nerve; left brain cerebrum, lateral geniculate nucleus (LGN), optic chiasm, optic tract, and visual cortex.

RNA Preparation from Tissue Specimens

RNA was extracted from approximately 25 mg of tissue samples (except spleen, which was ~10 mg) using the Zymo Quick-RNA™ Miniprep Plus Kit. The concentrations of the extracted RNA samples were measured using a Cytation 5 Imaging Reader via Quant-it™ RiboGreen RNA Assay Kit (Thermo Fisher Scientific). The extracted RNA samples were processed with TURBO DNA-Free™ Kit (Thermo Fisher Scientific) treatment and stored at −80° C. before RT-qPCR analysis.

### Standard Curve Samples

A Standard Curve dilution series was included in each Human CFI mRNA assay reaction plate. The standard curve dilutions were prepared using the control RNA at the concentrations listed in Table 3. All the standard curve dilutions consisted of 500 ng of NHP liver RNA matrix and were run in duplicate per reaction.

TABLE 3
Preparation of Human CFI mRNA Assay Standard Curve

| | Control RNA | Standard Matrix |
|---|---|---|
| Standard # | (copies/reaction) | gRNA (ng/reaction) |
| Std1 | 5E7 | 500 |
| Std2 | 5E6 | 500 |
| Std3 | 5E5 | 500 |
| Std4 | 5E4 | 500 |
| Std5 | 1E4 | 500 |
| Std6 | 2000 | 500 |
| Std7 | 400 | 500 |
| Std8 | 100 | 500 |

### No Template Controls (NTC)

No Template Controls (NTC) were included in each qPCR plate in triplicate wells as a negative control to assess potential contamination of the reaction components. The diluent, PolyA/TE buffer, was used as the NTC.

### Negative Control Samples

Negative controls consisting of 500 ng of NHP liver RNA matrix (NEG) were prepared and run in triplicate wells. Certain samples also contained a no-reverse transcriptase (no-RT) control.

### Spike Control

RNA generated from the control RNA was used to assess PCR inhibition. 500,000 copies per reaction of the control RNA were spiked into the third-well of each sample. Duplicate spike control wells with 500,000 copies per reaction of the control RNA and 500 ng of naïve NHP liver RNA were also included in each RT-qPCR plate.

### Assay Control

The assay control (AC) sample consisting of 500 ng of naïve NHP liver RNA and 1,000,000 copies of control RNA was included in each RT-qPCR plate in triplicate wells to test the assay's performance. The prepared AC samples were aliquoted and stored at −80° C.

### Data Analysis

After each RT-qPCR run, the data were analyzed using QuantStudio™ Design and Analysis Software v 2.5.0 (Thermo Fisher Scientific) and the Cₜ values of each reaction were determined with a Cₜ threshold set in the exponential phase of amplification. The copies of target RNA (Mean Quantity) were calculated by averaging the quantities detected in the first two wells for each sample. The Cₜ values of the third well sample containing a spike were examined to determine whether the Cₜ value of each third well was one Cₜ (≥1 Cₜ) higher than the spike control to assess PCR inhibition. The number of target copies per reaction, if equal to or above the LOQ (100 copies/reaction), was calculated and reported. Samples with measured quantity below the LOQ are reported as <LOQ.

Each reaction plate was evaluated according to the following acceptance criteria:

Target amplification is detected in the standard curve by qPCR for the target assay with a coefficient of determination (R²)≥0.99 and a slope of −3.1 to −3.6.

For each standard, the difference between the duplicate Cₜ values must be less than 1.0.

For the 8-point standard curve, up to two outlier points may be excluded from calculation to improve the standard curve slope and fit for more accurate quantitation, with the exception of Std1 and Std8. The average Cₜ for the NTC and NEG must be greater than the average Cₜ of the LOD (25 copies per reaction) as determined by the assay development.

The Cₜ values of replicate wells for each test sample must be within 1 Cₜ of each other, if within the qualified standard curve range of quantitation (100 copies/reaction to 5E7 copies/reaction), to be qualified for test sample quantitation calculation.

The Cₜ values of the third well test sample with RNA spike must be no more than one Cₜ value greater than the spike control Cₜ value to be considered to have uninhibited PCR. All sample test results met the criteria listed above. The sample test results are valid.

### Testing Results

RT-qPCR data were evaluated according to the acceptance criteria above. RT-qPCR plates used for the project data analysis had a qualified standard curve dilution point Std8 (the LOQ) with replicate reactions differing by less than 1 Cₜ value, thereby meeting the acceptance criteria. All the qualified plates had their standard curve slopes within −3.1 to −3.6 and coefficients of determination (R²) greater than 0.99, meeting the acceptance criteria. Sufficient RNA was tested for 143 samples at 400-500 ng/reaction. There were 150 samples that were tested at an RNA amount from 1.522 ng to 399.75 ng/reaction. The test results samples were reported as the number of human CFI mRNA copies detected per microgram (μg) of matrix RNA tested (copies/1 μg RNA). For samples with a quantity of human CFI mRNA detected below the LOQ, “<LOQ” is reported. For samples with human CFI copies≥LOQ, copies of the human CFI mRNA were quantified. 3 samples were retested due to duplicate wells being greater than 1 Cₜ value apart and 36 samples were retested due to qPCR inhibition.

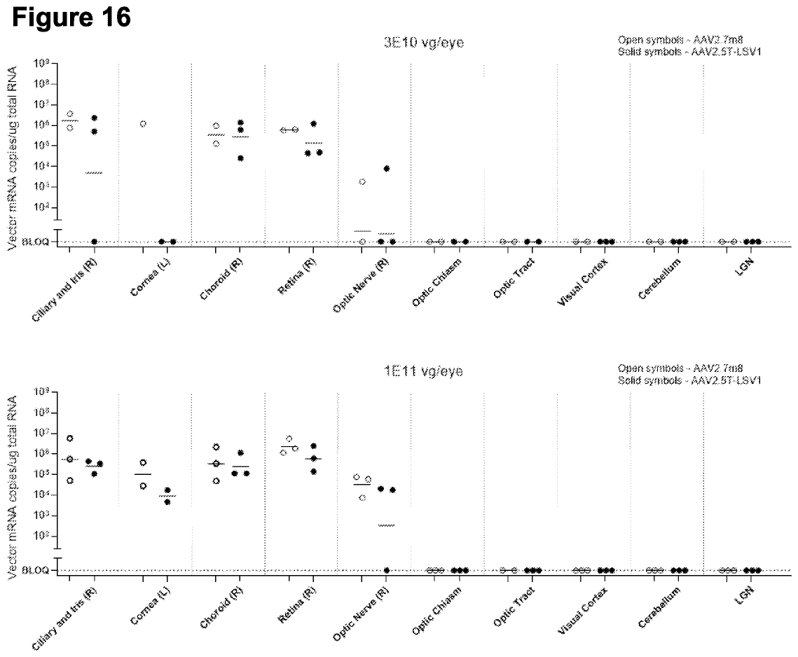

The data set obtained in this example is represented in FIG. 16.

What is claimed is:

1. A polynucleotide cassette for increasing the concentration of human CFI in the vitreous humor and/or retinal extracellular space of a mammalian eye, comprising:
a. an enhancer and promoter region;
b. a chimeric intron;
c. a Kozak sequence
d. a unique coding sequence optimized for increased expression and low CpG, operatively linked to the promoter region wherein the coding sequence is a human CFI gene,
e. a human scaffold attachment region; and
f. a polyadenylation site.

2. The polynucleotide cassette of claim 1, wherein the promoter region further comprises an enhancer element.

3. The polynucleotide cassette of claim 1, wherein the chimeric intron comprises at least one element selected from the group comprising: (a) an adenovirus tripartite leader sequence (TPL), (b) an enhancer element, and (c) an intron from mouse IgH.

4. The polynucleotide cassette of claim 1, further comprising AAV2 inverted terminal repeats (ITRs).

5. The polynucleotide cassette of claim 1, wherein the polyadenylation site is the human growth hormone polyadenylation site.

6. The polynucleotide cassette of claim 1, wherein the coding sequence has a nucleotide sequence having at least 90% identity to the nucleotide sequence set forth in SEQ ID NO:1.

7. The polynucleotide cassette of claim 1, wherein the coding sequence of the human CFI gene is selected from the group consisting of SEQ ID NO:2, SEQ ID NO:3, SEQ ID NO:4, SEQ ID NO:5, and SEQ ID NO:6.

8. A method of increasing the concentration of human CFI in the vitreous humor and/or retinal extracellular space of a mammalian eye, comprising administering to a subject a recombinant adeno-associated virus (rAAV) vector at a dosage ranging from about 1E⁹ to about 4E¹² vector genomes (vg)/eye, wherein the rAAV vector comprises an AAV2 capsid variant and wherein the rAAV vector comprises a polynucleotide cassette comprising:
a. An enhancer and promoter region;
b. a chimeric intron;
c. a Kozak sequence
d. a unique coding sequence optimized for increased expression and low CpG, operatively linked to the promoter region wherein the coding sequence is a human CFI gene,
e. a human scaffold attachment region; and
f. a polyadenylation site.

9. The method of claim 8 wherein the polynucleotide cassette further comprises an enhancer element.

10. The method of claim 8, wherein the chimeric intron of the polynucleotide cassette comprises at least one element selected from the group comprising: (a) an adenovirus tripartite leader sequence (TPL), (b) an enhancer element, and (c) an intron from mouse IgH.

11. The method of claim 8, wherein the polynucleotide cassette further comprises AAV2 inverted terminal repeats (ITRs).

12. The method of claim 8, wherein the polyadenylation site of the polynucleotide cassette is the human growth hormone polyadenylation site.

13. The method of claim 8, wherein the polynucleotide cassette has a nucleotide sequence having at least 90% identity to the nucleotide sequence set forth in SEQ ID NO:1.

14. The method of claim 8, wherein the coding sequence of a human CFI gene is selected from the group consisting of SEQ ID NO:2, SEQ ID NO:3, SEQ ID NO:4, SEQ ID NO:5 and SEQ ID NO:6.

15. The method of claim 8, wherein rAAV vector is administered intravitreally.

16. The method of claim 8, wherein the AAV2 capsid variant is selected from the group comprising AAV2.7m8 and AAV2.5T-LSV1.

17. The method of claim 8, wherein the rAAV vector is administered at a dosing range selected from the group of ranges consisting of from about 1E⁹ to about 4E¹² vg/eye, from about 10E¹⁰ to about 4E¹¹ vg/eye, from about 2E¹⁰ to about 3E¹¹ vg/eye, from about 2E¹⁰ to about 2E¹¹ vg/eye, from about 2.5E¹⁰ to about 2E¹¹ vg/eye, from about 2E¹⁰ to about 1E¹¹ vg/eye, from about 2E¹⁰ to about 9E¹⁰ vg/eye, from about 2E¹⁰ to about 8E¹⁰ vg/eye, and from about 3E¹⁰ to about 7E¹⁰ vg/eye.

18. The method of claim 8, wherein human CFI is detectable in the vitreous humor at least 4 weeks after intravitreal injection of said rAAV vector.

19. The method of claim 8, wherein the level of C3b-inactivating activity in an eye of said subject is increased.

20. The method of claim 8, wherein the level of iC3b-degradation activity in an eye of the subject is increased.

21. The method of claim 8, wherein the rAAV vector is administered as an intravitreal injection.

22. The method of claim 8, wherein the human CFI expressed from the rAAV vector is capable of degrading C3b into iC3b.

23. A polynucleotide cassette for increasing the concentration of human CFI in a mammalian eye, comprising:
a. an enhancer and promoter region;
b. a chimeric intron;
c. a Kozak sequence
d. a unique coding sequence optimized for increased expression and low CpG, operatively linked to the promoter region wherein the coding sequence is a human CFI gene,
e. a human scaffold attachment region; and
f. a polyadenylation site.

24. The polynucleotide cassette of claim 24, wherein said polynucleotide cassette is for increasing the concentration of human CFI in the vitreous humor and/or retinal extracellular space of a mammalian eye.

25. A method of treating dry acute macular degeneration (dry-AMD) in a human subject, comprising administering to a subject in need thereof a therapeutically effective amount of recombinant adeno-associated virus (rAAV) vector, wherein the rAAV vector comprises an AAV2 capsid variant and a polynucleotide cassette comprising:
a. An enhancer and promoter region;
b. a chimeric intron;
c. a Kozak sequence
d. a unique coding sequence optimized for increased expression and low CpG, operatively linked to the promoter region wherein the coding sequence is a human CFI gene,
e. a human scaffold attachment region; and
f. a polyadenylation site.

26. The method of claim 26, wherein the rAAV vector is administered intravitreally.

27. The method of claim 26, wherein the therapeutically effective amount is a dosage ranging from about 1E⁹ to about 4E¹² vector genomes (vg)/eye.

28. The method of claim 26, wherein there is a reduction in the geographic atrophy area and/or in the rate of growth of the geographic atrophy area after administration of the rAAV vector.

29. The method of claim 26, wherein the therapeutically effective amount is a dosage ranging from about 2E¹⁰ to about 9E¹⁰ vector genomes (vg)/eye.

30. An intravitreal dosage form, comprising a recombinant adeno-associated virus (rAAV) vector at a dosage ranging from about 1E⁹ to about 4E¹² vg/eye, wherein the rAAV vector comprises a polynucleotide cassette according to claim 1, and wherein the rAAV vector comprises an AAV2 capsid variant.

31. The intravitreal dosage form of claim 30, wherein rAAV vector comprises an AAV2_7m8 capsid variant and a dosage ranging from about 9E⁹ to about 9E¹⁰ vg/eye.


SEQUENCE LISTING (15 sequences)

SEQ ID NO: 1 - DNA, 4291 bp, synthetic construct
  source 1..4291: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagaggccatccacgctgttttgacctccatagtggacaccgggaccgatccagcctccgcgtctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccaccatgaagctcctgcatgtgtttctcctgttcctctgcttccacttgagattctgcaaggtcacctacaccagccaggaggacctggtggagaagaagtgcctggccaagaagtacactcacctgagctgtgacaaagtgttctgccagccttggcagagatgcatagagggaacctgtgtctgcaagctgccctaccagtgccctaagaatggcactgcagtctgtgccaccaacagaagatccttccccacctactgccagcagaagtcccttgagtgcctccaccctggcaccaagttcctgaacaacggcacttgcacagctgagggaaagttctcagtgtccctgaaacatggcaacactgactcagagggcattgtggaagtcaagcttgtggaccaggacaagaccatgttcatctgcaagtcctcctggtccatgcgggaagccaatgtggcctgccttgacttgggattccagcagggtgcagacacccagagaaggttcaagctgtcagacctcagcatcaactccactgaatgcctgcatgtgcactgccggggtctggaaacctccctggctgagtgcaccttcaccaagcgcaggaccatgggctaccaagactttgctgatgtggtttgctacacccagaaggcagactcccccatggatgacttcttccaatgtgtcaacggaaagtacatcagccagatgaaggcctgtgatggaatcaatgactgtggggaccagtcagatgaactgtgctgcaaggcctgccaggggaagggtttccactgcaagagtggagtgtgcatcccctcccaataccagtgcaatggagaagtggactgcatcactggggaagatgaagtgggctgtgcaggctttgcctctgtgacccaagaagaaactgagatcctgactgctgacatggatgcggagaggcgcaggatcaagtccctgctgccaaagctgtcctgtggtgtcaagaacagaatgcacatcaggcgcaagaggattgtgggagggaagagagcccagttgggggacctcccttggcaagtggccatcaaggatgcctcaggaatcacctgtggtggcatctacattggtggctgctggatcctcactgctgcccactgcctgagggccagcaaaacccacagataccagatctggaccactgtggtggactggatccacccagacctgaagcggattgtgattgaatatgtggaccgcatcatcttccatgagaactacaatgcaggcacctaccagaatgacattgccctgattgagatgaagaaggatggaaacaagaaagactgtgaactccctcggagcatcccagcctgtgtgccatggtccccctacctgttccaacccaatgacacttgcattgtgtcaggctggggcagagaaaaggacaatgagcgggtgttcagcctgcaatggggagaggtcaagctcatctccaactgctccaagttctatgggaacagattctatgaaaaggaaatggagtgtgctggcacctatgatgggagcattgatgcctgcaaaggagactctgggggacccctggtctgcatggatgccaacaatgtcacctatgtctggggagtggtgtcctggggagaaaactgtggaaagcctgagttccctggagtgtacaccaaagtggccaactactttgactggatctcctaccatgtgggaaggcccttcattagccagtacaacgtgtaataactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 2 - DNA, 1752 bp, synthetic construct
  source 1..1752: mol_type = other DNA
atgaagctgctgcatgtgttcctcctgtttctgtgcttccatctgagattctgcaaggtcacctatacttcccaagaggatctggtggagaaaaagtgcctggccaagaagtacacccacctgtcgtgtgataaggtcttttgccaaccgtggcagcggtgcatcgaaggcacttgcgtgtgcaagctgccgtaccagtgtccgaagaatggaaccgcggtctgtgcaaccaatagacgctccttccccacctactgtcagcagaagtccctggagtgcctgcacccgggtactaagttcctcaacaacggaacgtgcaccgccgaggggaagttctccgtgagccttaaacacgggaacaccgattcagagggtatagtggaagtcaagctggtcgatcaggacaagactatgtttatttgcaagtcctcgtggtccatgagagaagccaacgtggcctgcctggacctgggattccagcagggtgccgatacccagaggcggttcaagctgtccgacctgtcaatcaactcgactgagtgcttgcacgtgcactgccggggcctcgaaacctcgctggccgaatgcactttcaccaagcggcgcacaatgggttaccaggacttcgcagacgtcgtgtgctacacccaaaaggccgattcccccatggatgacttcttccaatgcgtgaacgggaagtacatcagccagatgaaggcctgcgatggcatcaacgactgtggggaccagtcggacgaactgtgctgcaaggcctgccagggaaagggcttccactgcaagagcggggtctgcatcccgtcacaataccagtgcaatggcgaagtggactgtatcaccggagaggacgaggtcggctgtgcgggttttgccagcgttacccaggaagaaaccgagatcctcactgccgacatggacgccgagcgcagacgcatcaagtccttgctgccaaagctctcgtgcggagtgaagaaccgcatgcacatccgccggaagcgcattgtgggcggaaagagagctcagctcggagacttaccttggcaagtggccatcaaggacgcttctggcattacttgcgggggcatctacattggcggctgctggattctgaccgcggcccattgcctgagggcttcaaaaactcataggtaccagatctggaccacggtagtggactggattcaccccgacctcaagcggatcgtgatcgaatacgtggacaggatcatcttccacgagaactataacgccggaacctaccagaacgatatcgcgctgatcgagatgaagaaggacggaaacaagaaagactgtgaacttccccggtccatccctgcttgcgtgccctggtccccttacttgttccaaccgaacgacacttgtatcgtgtccggatggggccgggaaaaagacaacgaacgcgtgttcagcctgcaatggggagaagtgaaactcatctccaactgctccaagttctacggcaaccgattctacgaaaaggaaatggagtgtgccggcacctacgacggatcgattgacgcgtgcaaaggggatagcggcgggcctcttgtgtgcatggatgcaaacaacgtgacctacgtctggggcgtggtgtcctggggagagaactgtggaaagcccgagttcccgggagtgtacaccaaagtggcgaactatttcgattggatttcataccacgtcggacggccattcattagccagtacaatgtgtaa

SEQ ID NO: 3 - DNA, 1752 bp, synthetic construct
  source 1..1752: mol_type = other DNA
atgaagctgctgcatgtgttcctcctgttcctgtgcttccacctgagattctgcaaggtcacctacaccagccaggaagatctggtggagaagaagtgcctggccaagaagtacacccacttgagctgtgacaaggtcttttgccaaccctggcagcgctgcatagaaggcacctgtgtctgcaagctgccgtaccagtgcccgaagaacggcactgctgtgtgtgccaccaaccgaagaagcttcccaacctactgccagcagaagtccttggagtgcctgcaccctggcaccaagttcctgaacaacggcacctgtactgcggagggaaagttctcagtgtccctgaagcatggaaacactgactcagagggcattgtggaagtgaaacttgtggaccaggacaagaccatgttcatctgcaagtccagctggagcatgagagaggccaacgtggcctgcctggacctggggttccagcagggtgcagacactcagcgccggttcaagctgtcagacctctccatcaactccactgaatgcctgcatgtgcactgccggggactggaaaccagcctggctgagtgcaccttcaccaagagaagaaccatgggttaccaggactttgcagatgtggtctgctacacccagaaggcagactccccaatggatgacttcttccaatgtgtcaatgggaagtacatcagccagatgaaggcctgtgatggtatcaatgactgtggggaccagtcggatgagctgtgctgcaaggcctgccaggggaagggtttccactgcaaatcgggagtctgcatcccctcccaataccagtgcaatggagaagtggactgcattactggagaggatgaggttggctgtgctggatttgcctcagtgacccaggaagaaacagaaatcttgactgcggacatggatgctgaacggcgcaggatcaagtccctcctccccaaactcagctgtggagtgaagaaccggatgcacatcagacggaagcggattgtgggaggaaagagagcccagctgggggaccttccttggcaagtggccatcaaggatgcctctggcatcacctgtggtggcatctacattgggggatgctggatcctcactgctgcccactgcctgagggcctccaagacccaccggtaccagatctggaccactgtggtggactggatccaccctgacctgaagaggattgtgattgaatatgtggacaggatcatcttccatgaaaactacaatgcaggcacctaccagaacgacattgccctcattgagatgaagaaggatggcaacaagaaggactgtgaacttccccgctccatcccggcctgtgtgccctggtccccctacctgttccaacctaatgacacttgcattgtgtcaggctggggaagagagaaggacaatgagagggtgttctccctccaatggggagaagtcaagctcatctccaactgctccaagttctatggcaaccgcttctatgagaaagaaatggaatgtgcaggaacttatgatggttccattgatgcctgcaaaggggacagtggtggcccactggtctgcatggatgccaacaacgtgacctatgtctggggagtggtgtcctggggagagaactgtgggaagcctgagttccctggagtgtacaccaaagtggccaactactttgactggatcagctaccatgtgggcaggcccttcatctcccaatacaatgtctaa

SEQ ID NO: 4 - DNA, 1752 bp, synthetic construct
  source 1..1752: mol_type = other DNA
atgaagctcctgcatgtgtttctcctgttcctctgcttccacttgagattctgcaaggtcacctacaccagccaggaggacctggtggagaagaagtgcctggccaagaagtacactcacctgagctgtgacaaagtgttctgccagccttggcagagatgcatagagggaacctgtgtctgcaagctgccctaccagtgccctaagaatggcactgcagtctgtgccaccaacagaagatccttccccacctactgccagcagaagtcccttgagtgcctccaccctggcaccaagttcctgaacaacggcacttgcacagctgagggaaagttctcagtgtccctgaaacatggcaacactgactcagagggcattgtggaagtcaagcttgtggaccaggacaagaccatgttcatctgcaagtcctcctggtccatgcgggaagccaatgtggcctgccttgacttgggattccagcagggtgcagacacccagagaaggttcaagctgtcagacctcagcatcaactccactgaatgcctgcatgtgcactgccggggtctggaaacctccctggctgagtgcaccttcaccaagcgcaggaccatgggctaccaagactttgctgatgtggtttgctacacccagaaggcagactcccccatggatgacttcttccaatgtgtcaacggaaagtacatcagccagatgaaggcctgtgatggaatcaatgactgtggggaccagtcagatgaactgtgctgcaaggcctgccaggggaagggtttccactgcaagagtggagtgtgcatcccctcccaataccagtgcaatggagaagtggactgcatcactggggaagatgaagtgggctgtgcaggctttgcctctgtgacccaagaagaaactgagatcctgactgctgacatggatgcggagaggcgcaggatcaagtccctgctgccaaagctgtcctgtggtgtcaagaacagaatgcacatcaggcgcaagaggattgtgggagggaagagagcccagttgggggacctcccttggcaagtggccatcaaggatgcctcaggaatcacctgtggtggcatctacattggtggctgctggatcctcactgctgcccactgcctgagggccagcaaaacccacagataccagatctggaccactgtggtggactggatccacccagacctgaagcggattgtgattgaatatgtggaccgcatcatcttccatgagaactacaatgcaggcacctaccagaatgacattgccctgattgagatgaagaaggatggaaacaagaaagactgtgaactccctcggagcatcccagcctgtgtgccatggtccccctacctgttccaacccaatgacacttgcattgtgtcaggctggggcagagaaaaggacaatgagcgggtgttcagcctgcaatggggagaggtcaagctcatctccaactgctccaagttctatgggaacagattctatgaaaaggaaatggagtgtgctggcacctatgatgggagcattgatgcctgcaaaggagactctgggggacccctggtctgcatggatgccaacaatgtcacctatgtctggggagtggtgtcctggggagaaaactgtggaaagcctgagttccctggagtgtacaccaaagtggccaactactttgactggatctcctaccatgtgggaaggcccttcattagccagtacaacgtgtaa

SEQ ID NO: 5 - DNA, 1752 bp, synthetic construct
  source 1..1752: mol_type = other DNA
atgaaacttctgcatgtgttcctgctgttcctctgcttccacctgagattctgcaaggtcacctacacctcccaagaggacctggtggagaagaagtgcctggccaagaagtacacccacctgtcctgtgacaaggtcttttgccaaccatggcagagatgcattgaagggacctgtgtctgcaagctgccctaccaatgccccaagaatggtactgcagtctgtgccaccaaccggcgctccttccccacctactgccagcagaagtccctggaatgcctccaccctggcaccaagttcctgaacaatggcacttgcactgctgagggaaagttctctgtgtccctcaagcatgggaacactgactcagagggcattgtggaagtgaaactggtggaccaggacaagactatgttcatctgcaagtcctcctggtccatgagagaggccaatgtggcctgcctggacctggggttccagcagggtgcagacacccagaggagattcaaactgtcagacctcagcatcaactccactgaatgcctgcatgtgcactgcagaggactggagacttccctggctgagtgcaccttcaccaagcgcaggaccatgggctaccaggactttgctgatgtggtctgctacacccagaaggcagactcccccatggatgacttcttccaatgtgtaaatggaaagtacatcagccagatgaaggcctgtgatggtatcaatgactgtggggaccagtcagatgaactgtgctgcaaggcctgccagggaaagggcttccactgcaaatctggagtgtgcatcccaagccagtaccagtgcaacggggaagtggactgcatcactggagaggatgaagtgggctgtgcaggatttgcctcagtgacccaggaggaaactgaaatcttgactgctgatatggatgctgagagaagaaggatcaagtccctgctgccaaagctctcctgtggagtgaagaacagaatgcacatcaggagaaagagaattgtgggaggaaagagagcccagctgggagacttgccttggcaagtggccataaaggatgcctcaggaatcacctgtggtggcatctacattggtggctgctggatcctgacagctgcccactgcctgagggcctccaagacccacagataccagatctggaccactgtggtggactggatccaccctgacttgaagaggattgtgattgaatatgtggacaggatcatcttccatgaaaactacaatgctggaacctaccagaatgacattgcccttattgagatgaagaaggatgggaacaagaaggactgtgaactcccccggagcatccctgcctgtgtgccctggagcccctacctcttccaaccaaatgacacttgcattgtgtcaggctggggccgggagaaagacaatgagagggtgttcagccttcaatggggagaagtcaagctcatctccaactgctccaagttctatggcaacagattctatgaaaaggagatggaatgtgcaggcacctatgatggcagcattgatgcctgcaagggggacagtggtggccctctggtctgcatggatgccaacaatgtcacctatgtctggggagtggtcagctggggagaaaactgtgggaagcctgagttccctggagtgtacaccaaagtggccaactactttgactggatctcctaccatgttggaaggcccttcatcagccagtacaatgtgtaa

SEQ ID NO: 6 - DNA, 1752 bp, synthetic construct
  source 1..1752: mol_type = other DNA
atgaagctcctgcatgtgtttctcctcttcctgtgcttccacctgagattctgcaaggtcacctacaccagccaggaggacctggtggaaaagaagtgcctggccaagaagtacacccacctgtcctgtgacaaagtgttctgccaaccctggcaaagatgcattgaaggcacctgtgtctgcaagctgccctaccagtgccccaagaatggtactgctgtgtgtgccactaacagaaggtccttcccaacctactgccagcagaagtccttggaatgccttcaccctgggaccaagttcctcaacaatggcacctgtactgctgagggaaagttctcagtgtccctgaagcatggcaacacagactcagagggcattgtggaggtcaagcttgtggaccaggacaagaccatgttcatctgcaagtccagctggagcatgagagaggccaatgtggcctgcctggacctgggattccagcagggtgcagacacccagagaaggttcaagttgtcagacctctccatcaactccactgagtgcctccatgtgcactgcagagggctggaaacaagcttggcagaatgcaccttcaccaagaggaggaccatgggctaccaggactttgcagatgtggtctgctacacccaaaaggcagacagtcccatggatgacttcttccaatgtgtcaatgggaagtacatcagccagatgaaggcctgtgatggtatcaatgactgtggggaccagtcagatgaactgtgctgcaaggcctgccagggcaaagggttccactgcaaatcaggggtctgcatcccaagccagtaccagtgcaatggagaagtggactgcatcactggagaggatgaagttggatgtgctggatttgcctcagtgacccaggaagagactgaaatcctcactgctgacatggatgctgagagaagaagaatcaagtccctgctgcccaagctgagctgtggagtgaagaacaggatgcacatcagaaggaagagaattgtgggagggaaaagggcccagctgggtgatctcccttggcaagtggccatcaaggatgcctcaggaatcacctgtggaggcatctacattggtggctgctggatcctgactgctgcccactgcctgagagcctccaagacccacagataccagatctggaccactgtggtggactggatccaccctgaccttaagaggattgtgattgaatatgtggacagaatcatcttccatgagaactacaatgctggcacctaccagaatgacattgccctgattgagatgaagaaggatggcaacaagaaggactgtgaactgcctaggtccatccctgcctgtgtgccctggtccccctacctcttccaacctaatgacacttgcattgtgtctggatggggcagagagaaggacaatgagagggtgttctccctgcaatggggagaagtcaagctgatctccaactgctccaagttctatggcaacagattctatgaaaaagaaatggagtgtgcagggacctatgatggctccatagatgcctgcaagggagactctggtggcccactggtctgcatggatgccaacaatgtcacctatgtctggggagtggtgtcctggggagaaaactgtggaaagccagagttccctggagtgtacaccaaagtggccaactactttgactggatctcctaccatgtgggaaggcccttcattagccagtacaatgtgtaa

SEQ ID NO: 7 - DNA, 145 bp, synthetic construct
  source 1..145: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaac

SEQ ID NO: 8 - DNA, 585 bp, synthetic construct
  source 1..585: mol_type = other DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagaggccatccacgctgttttgacctccatagtggacaccgggaccgatccagcctccg

SEQ ID NO: 9 - DNA, 206 bp, synthetic construct
  source 1..206: mol_type = other DNA
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaag

SEQ ID NO: 10 - DNA, 238 bp, synthetic construct
  source 1..238: mol_type = other DNA
gtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccg

SEQ ID NO: 11 - DNA, 54 bp, synthetic construct
  source 1..54: mol_type = other DNA
atctggccatacacttgagtgacaatgacatccactttgcctttctctccacag

SEQ ID NO: 12 - skipped

SEQ ID NO: 13 - DNA, 810 bp, synthetic construct
  source 1..810: mol_type = other DNA
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 14 - DNA, 202 bp, synthetic construct
  source 1..202: mol_type = other DNA
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 15 - DNA, 145 bp, synthetic construct
  source 1..145: mol_type = other DNA
gttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc